SEQUENCE LISTING (38 sequences)

SEQ ID NO: 1 - protein, 609 aa, Homo sapiens
MKWVESIFLIFLLNFTESRTLHRNEYGIASILDSYQCTAEISLADLATIFFAQFVQEATYKEVSKMVKDALTAIEKPTGDEQSSGCLENQLPAFLEELCHEKEILEKYGHSDCCSQSEEGRHNCFLAHKKPTPASIPLFQVPEPVTSCEAYEEDRETFMNKFIYEIARRHPFLYAPTILLWAARYDKIIPSCCKAENAVECFQTKAATVTKELRESSLLNQHACAVMKNFGTRTFQAITVTKLSQKFTKVNFTEIQKLVLDVAHVHEHCCRGDVLDCLQDGEKIMSYICSQQDTLSNKITECCKLTTLERGQCIIHAENDEKPEGLSPNLNRFLGDRDFNQFSSGEKNIFLASFVHEYSRRHPQLAVSVILRVAKGYQELLEKCFQTENPLECQDKGEEELQKYIQESQALAKRSCGLFQKLGEYYLQNAFLVAYTKKAPQLTSSELMAITRKMAATAATCCQLSEDKLLACGEGAADIIIGHLCIRHEMTPVNPGVGQCCTSSYANRRPCFSSLVVDETYVPPAFSDDKFIFHKDLCQAQGVALQTMKQEFLINLVKQKPQITEEQLEAVIADFSGLLEKCCQGQEQEVCFAEEGQKLISKTRAALGV

SEQ ID NO: 2 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 3 - protein, 1443 aa, Homo sapiens
MYAVYKQAHPPTGLEFSMYCNFFNNSERNLVVAGTSQLYVYRLNRDAEALTKNDRSTEGKAHREKLELAASFSFFGNVMSMASVQLAGAKRDALLLSFKDAKLSVVEYDPGTHDLKTLSLHYFEEPELRDGFVQNVHTPRVRVDPDGRCAAMLVYGTRLVVLPFRRESLAEEHEGLVGEGQRSSFLPSYIIDVRALDEKLLNIIDLQFLHGYYEPTLLILFEPNQTWPGRVAVRQDTCSIVAISLNITQKVHPVIWSLTSLPFDCTQALAVPKPIGGVVVFAVNSLLYLNQSVPPYGVALNSLTTGTTAFPLRTQEGVRITLDCAQATFISYDKMVISLKGGEIYVLTLITDGMRSVRAFHFDKAAASVLTTSMVTMEPGYLFLGSRLGNSLLLKYTEKLQEPPASAVREAADKEEPPSKKKRVDATAGWSAAGKSVPQDEVDEIEVYGSEAQSGTQLATYSFEVCDSILNIGPCANAAVGEPAFLSEEFQNSPEPDLEIVVCSGHGKNGALSVLQKSIRPQVVTTFELPGCYDMWTVIAPVRKEEEDNPKGEGTEQEPSTTPEADDDGRRHGFLILSREDSTMILQTGQEIMELDTSGFATQGPTVFAGNIGDNRYIVQVSPLGIRLLEGVNQLHFIPVDLGAPIVQCAVADPYVVIMSAEGHVTMFLLKSDSYGGRHHRLALHKPPLHHQSKVITLCLYRDLSGMFTTESRLGGARDELGGRSGPEAEGLGSETSPTVDDEEEMLYGDSGSLFSPSKEEARRSSQPPADRDPAPFRAEPTHWCLLVRENGTMEIYQLPDWRLVFLVKNFPVGQRVLVDSSFGQPTTQGEARREEATRQGELPLVKEVLLVALGSRQSRPYLLVHVDQELLIYEAFPHDSQLGQGNLKVRFKKVPHNINFREKKPKPSKKKAEGGGAEEGAGARGRVARFRYFEDIYGYSGVFICGPSPHWLLVTGRGALRLHPMAIDGPVDSFAPFHNVNCPRGFLYFNRQGELRISVLPAYLSYDAPWPVRKIPLRCTAHYVAYHVESKVYAVATSTNTPCARIPRMTGEEKEFETIERDERYIHPQQEAFSIQLISPVSWEAIPNARIELQEWEHVTCMKTVSLRSEETVSGLKGYVAAGTCLMQGEEVTCRGRILIMDVIEVVPEPGQPLTKNKFKVLYEKEQKGPVTALCHCNGHLVSAIGQKIFLWSLRASELTGMAFIDTQLYIHQMISVKNFILAADVMKSISLLRYQEESKTLSLVSRDAKPLEVYSVDFMVDNAQLGFLVSDRDRNLMVYMYLPEAKESFGGMRLLRRADFHVGAHVNTFWRTPCRGATEGLSKKSVVWENKHITWFATLDGGIGLLLPMQEKTYRRLLMLQNALTTMLPHHAGLNPRAFRMLHVDRRTLQNAVRNVLDGELLNRYLYLSTMERSELAKKIGTTPDIILDDLLETDRVTAHF

SEQ ID NO: 4 - protein, 459 aa, Homo sapiens
MAPLCPSPWLPLLIPAPAPGLTVQLLLSLLLLMPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 5 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGDTKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 6 - protein, 180 aa, Homo sapiens
MQAEGQGTGGSTGDADGPGGPGIPDGPGGNAGGPGEAGATGGRGPRGAGAARASGPRGGAPRGPHGGAASAQDGRCPCGARRPDSRLLQLHITMPFSSPMEAELVRRILSRDAAPLPRPGAVLKDFTVSGNLLFIRLTAADHRQLQLSISSCLQQLSLLMWITQCFLPVFLAQAPSGQRR

SEQ ID NO: 7 - protein, 369 aa, Homo sapiens
MPRAPKRQRCMPEEDLQSQSETQGLEGAQAPLAVEEDASSSTSTSSSFPSSFPSSSSSSSSSCYPLIPSTPEEVSADDETPNPPQSAQIACSSPSVVASLPLDQSDEGSSSQKEESPSTLQVLPDSESLPRSEIDEKVTDLVQFLLFKYQMKEPITKAEILESVIKNYEDHFPLLFSEASECMLLVFGIDVKEVDPTGHSFVLVTSLGLTYDGMLSDVQSMPKTGILILILSIIFIEGYCTPEEVIWEALNMMGLYDGMEHLIYGEPRKLLTQDWVQENYLEYRQVPGSDPARYEFLWGPRAHAEIRKMSLLKFLAKVNGSDPRSFPLWYEEALKDEEERAQDRIATTDDTTAMASASSSATGSFSYPE

SEQ ID NO: 8 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 9 - protein, 314 aa, Homo sapiens
MPLEQRSQHCKPEEGLEARGEALGLVGAQAPATEEQQTASSSSTLVEVTLGEVPAADSPSPPHSPQGASSFSTTINYTLWRQSDEGSSNQEEEGPRMFPDLESEFQAAISRKMVELVHFLLLKYRAREPVTKAEMLESVLRNCQDFFPVIFSKASEYLQLVFGIEVVEVVPISHLYILVTCLGLSYDGLLGDNQVMPKTGLLIIVLAIIAIEGDCAPEEKIWEELSMLEVFEGREDSVFAHPRKLLMQDLVQENYLEYRQVPGSDPACYEFLWGPRALIETSYVKVLHHTLKIGGEPHISYPPLHERALREGEE

SEQ ID NO: 10 - protein, 319 aa, Homo sapiens
MPRGQKSKLRAREKRRKARDETRGLNVPQVTEAEEEEAPCCSSSVSGGAASSSPAAGIPQKPQRAPTTAAAAAAGVSSTKSKKGAKSHQGEKNASSSQASTSTKSPSEDPLTRKSGSLVQFLLYKYKIKKSVTKGEMLKIVGKRFREHFPEILKKASEGLSVVFGLELNKVNPNGHTYTFIDKVDLTDEESLLSSWDFPRRKLLMPLLGVIFLNGNSATEEEIWEFLNMLGVYDGEEHSVFGEPWKLITKDLVQEKYLEYKQVPSSDPPRFQFLWGPRAYAETSKMKVLEFLAKVNGTTPCAFPTHYEEALKDEEKAGV

SEQ ID NO: 11 - protein, 317 aa, Homo sapiens
MAVQGSQRRLLGSLNSTPTAIPQLGLAANQTGARCLEVSISDGLFLSLGLVSLVENALVVATIAKNRNLHSPMYCFICCLALSDLLVSGSNVLETAVILLLEAGALVARAAVLQQLDNVIDVITCSSMLSSLCFLGAIAVDRYISIFYALRYHSTVTLPRARRAVAAIWVASVVFSTLFIAYYDHVAVLLCLVVFFLAMLVLMAVLYVHMLARACQHAQGIARLHKRQRPVHQGFGLKGAVTLTILLGIFFLCWGPFFLHLTLIVLCPEHPTCGCIFKNFNLFLALIICNAIIDPLIYAFHSQELRRTLKEVLTCSW

SEQ ID NO: 12 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 13 - protein, 273 aa, Homo sapiens
MTPGTQSPFFLLLLLTVLTVVTGSGHASSTPGGEKETSATQRSSVPSSTEKNALSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 14 - protein, 5179 aa, Homo sapiens
MGLPLARLAAVCLALSLAGGSELQTEGRTRNHGHNVCSTWGNFHYKTFDGDVFRFPGLCDYNFASDCRGSYKEFAVHLKRGPGQAEAPAGVESILLTIKDDTIYLTRHLAVLNGAVVSTPHYSPGLLIEKSDAYTKVYSRAGLTLMWNREDALMLELDTKFRNHTCGLCGDYNGLQSYSEFLSDGVLFSPLEFGNMQKINQPDVVCEDPEEEVAPASCSEHRAECERLLTAEAFADCQDLVPLEPYLRACQQDRCRCPGGDTCVCSTVAEFSRQCSHAGGRPGNWRTATLCPKTCPGNLVYLESGSPCMDTCSHLEVSSLCEEHRMDGCFCPEGTVYDDIGDSGCVPVSQCHCRLHGHLYTPGQEITNDCEQCVCNAGRWVCKDLPCPGTCALEGGSHITTFDGKTYTFHGDCYYVLAKGDHNDSYALLGELAPCGSTDKQTCLKTVVLLADKKKNVVVFKSDGSVLLNELQVNLPHVTASFSVFRPSSYHIMVSMAIGVRLQVQLAPVMQLFVTLDQASQGQVQGLCGNFNGLEGDDFKTASGLVEATGAGFANTWKAQSSCHDKLDWLDDPCSLNIESANYAEHWCSLLKKTETPFGRCHSAVDPAEYYKRCKYDTCNCQNNEDCLCAALSSYARACTAKGVMLWGWREHVCNKDVGSCPNSQVFLYNLTTCQQTCRSLSEADSHCLEGFAPVDGCGCPDHTFLDEKGRCVPLAKCSCYHRGLYLEAGDVVVRQEERCVCRDGRLHCRQIRLIGQSCTAPKIHMDCSNLTALATSKPRALSCQTLAAGYYHTECVSGCVCPDGLMDDGRGGCVVEKECPCVHNNDLYSSGAKIKVDCNTCTCKRGRWVCTQAVCHGTCSIYGSGHYITFDGKYYDFDGHCSYVAVQDYCGQNSSLGSFSIITENVPCGTTGVTCSKAIKIFMGRTELKLEDKHRVVIQRDEGHHVAYTTREVGQYLVVESSTGIIVIWDKRTTVFIKLAPSYKGTVCGLCGNFDHRSNNDFTTRDHMVVSSELDFGNSWKEAPTCPDVSTNPEPCSLNPHRRSWAEKQCSILKSSVFSICHSKVDPKPFYEACVHDSCSCDTGGDCECFCSAVASYAQECTKEGACVFWRTPDLCPIFCDYYNPPHECEWHYEPCGNRSFETCRTINGIHSNISVSYLEGCYPRCPKDRPIYEEDLKKCVTADKCGCYVEDTHYPPGASVPTEETCKSCVCTNSSQVVCRPEEGKILNQTQDGAFCYWEICGPNGTVEKHFNICSITTRPSTLTTFTTITLPTTPTTFTTTTTTTTPTSSTVLSTTPKLCCLWSDWINEDHPSSGSDDGDRETFDGVCGAPEDIECRSVKDPHLSLEQLGQKVQCDVSVGFICKNEDQFGNGPFGLCYDYKIRVNCCWPMDKCITTPSPPTTTPSPPPTSTTTLPPTTTPSPPTTTTTTPPPTTTPSPPITTTTTPPPTTTPSPPISTTTTPPPTTTPSPPTTTPSPPTTTPSPPTTTTTTPPPTTTPSPPTTTPITPPASTTTLPPTTTPSPPTTTTTTPPPTTTPSPPTTTPITPPTSTTTLPPTTTPSPPPTTTTTPPPTTTPSPPTTTTPSPPTITTTTPPPTTTPSPPTTTTTTPPPTTTPSPPTTTPITPPTSTTTLPPTTTPSPPPTTTTTPPPTTTPSPPTTTTPSPPITTTTTPPPTTTPSSPITTTPSPPTTTMTTPSPTTTPSSPITTTTTPSSTTTPSPPPTTMTTPSPTTTPSPPTTTMTTLPPTTTSSPLTTTPLPPSITPPTFSPFSTTTPTTPCVPLCNWTGWLDSGKPNFHKPGGDTELIGDVCGPGWAANISCRATMYPDVPIGQLGQTVVCDVSVGLICKNEDQKPGGVIPMAFCLNYEINVQCCECVTQPTTMTTTTTENPTPPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTPTTTPITTTTTVTPTPTPTGTQTGPPTHTSTAPIAELTTSNPPPESSTPQTSRSTSSPLTESTTLLSTLPPAIEMTSTAPPSTPTAPTTTSGGHTLSPPPSTTTSPPGTPTRGTTTGSSSAPTPSTVQTTTTSAWTPTPTPLSTPSIIRTTGLRPYPSSVLICCVLNDTYYAPGEEVYNGTYGDTCYFVNCSLSCTLEFYNWSCPSTPSPTPTPSKSTPTPSKPSSTPSKPTPGTKPPECPDFDPPRQENETWWLCDCFMATCKYNNTVEIVKVECEPPPMPTCSNGLQPVRVEDPDGCCWHWECDCYCTGWGDPHYVTFDGLYYSYQGNCTYVLVEEISPSVDNFGVYIDNYHCDPNDKVSCPRTLIVRHETQEVLIKTVHMMPMQVQVQVNRQAVALPYKKYGLEVYQSGINYVVDIPELGVLVSYNGLSFSVRLPYHRFGNNTKGQCGTCTNTTSDDCILPSGEIVSNCEAAADQWLVNDPSKPHCPHSSSTTKRPAVTVPGGGKTTPHKDCTPSPLCQLIKDSLFAQCHALVPPQHYYDACVFDSCFMPGSSLECASLQAYAALCAQQNICLDWRNHTHGACLVECPSHREYQACGPAEEPTCKSSSSQQNNTVLVEGCFCPEGTMNYAPGFDVCVKTCGCVGPDNVPREFGEHFEFDCKNCVCLEGGSGIICQPKRCSQKPVTHCVEDGTYLATEVNPADTCCNITVCKCNTSLCKEKPSVCPLGFEVKSKMVPGRCCPFYWCESKGVCVHGNAEYQPGSPVYSSKCQDCVCTDKVDNNTLLNVIACTHVPCNTSCSPGFELMEAPGECCKKCEQTHCIIKRPDNQHVILKPGDFKSDPKNNCTFFSCVKIHNQLISSVSNITCPNFDASICIPGSITFMPNGCCKTCTPRNETRVPCSTVPVTTEVSYAGCTKTVLMNHCSGSCGTFVMYSAKAQALDHSCSCCKEEKTSQREVVLSCPNGGSLTHTYTHIESCQCQDTVCGLPTGTSRRARRSPRHLGSG

SEQ ID NO: 15 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 16 - protein, 393 aa, Homo sapiens [sequence omitted]

SEQ ID NO: 17 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 18 - protein, 255 aa, Homo sapiens
MFSWTVVSLVLLTSIANLVGGFIVVRKEWSPKALTYLMAFSAGFLLSIGILDLMPEGLENSPENGIYILIGFLVLFSFQRILTTHFHFGYETHEDKLSKKTGGLGAFIGMTIHSFFDGVSIVAGFEVSSELGFLVFVAVLLHKIPDGLTISSIVLVVFNDRKKAFIASAVLALATIFGGALVWLLSDTEFAAEVLGDSFARIALSFSAGVFLYVAATDLLPVVNQSENRKTGLYVLLGVAVFYIASWIIGVVGLE

SEQ ID NO: 19 - protein, 1120 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 20 - protein, 519 aa, Homo sapiens
MSPLWWGFLLSCLGCKILPGAQGQFPRVCMTVDSLVNKECCPRLGAESANVCGSQQGRGQCTEVRADTRPWSGPYILRNQDDRELWPRKFFHRTCKCTGNFAGYNCGDCKFGWTGPNCERKKPPVIRQNIHSLSPQEREQFLGALDLAKKRVHPDYVITTQHWLGLLGPNGTQPQFANCSVYDFFVWLHYYSVRDTLLGPGRPYRAIDFSHQGPAFVTWHRYHLLCLERDLQRLIGNESFALPYWNFATGRNECDVCTDQLFGAARPDDPTLISRNSRFSSWETVCDSLDDYNHLVTLCNGTYEGLLRRNQMGRNSMKLPTLKDIRDCLSLQKFDNPPFFQNSTFSFRNALEGFDKADGTLDSQVMSLHNLVHSFLNGTNALPHSAANDPIFVVLHSFTDAIFDEWMKRFNPPADAWPQELAPIGHNRMYNMVPFFPPVTNEELFLTSDQLGYSYAIDLPVSVEETPGWPTTLLVVMGTLVALVGLFVLLAFLQYRRLRKGYTPLMETHLSSKRYTEEA

SEQ ID NO: 21 - protein, 529 aa, Homo sapiens [sequence omitted]

SEQ ID NO: 22 - protein, 500 aa, Homo sapiens
MQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 23 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 24 - protein, 1255 aa, Homo sapiens [sequence omitted]

SEQ ID NO: 25 - protein, 454 aa, Influenza A virus
MNPNQKITTIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTQGALLNDKHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 26 - protein, 230 aa, Influenza A virus
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 27 - protein, 252 aa, Influenza A virus
MSLLTEVETYVLSIIPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQMVTTTNPLIRHENRMVLASTTAKAMEQMAGSSEQAAEAMEVASQARQMVQAMRTIGTHPSSSAGLKNDLLENLQAYQKRMGVQMQRFK

SEQ ID NO: 28 - protein, 759 aa, Influenza A virus
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPITNTVHYPKIYKTYFERVERLKHGTFGPVHFRNQVKIRRRVDINPGHADLSAKEAQDVIMEVVFPNEVGARILTSESQLTITKEKKEELQDCKISPLMVAYMLERELVRKTRFLPVAGGTSSVYIEVLHLTQGTCWEQMYTPGGEVRNDDVDQSLIIAARNIVRRAAVSADPLASLLEMCHSTQIGGIRMVDILRQNPTEEQAVDICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGVEPIDNVMGMIGILPDMTPSIEMSMRGVRISKMGVDEYSSTERVVVSIDRFLRIRDQRGNVLLSPEEVSETQGTEKLTITYSSSMMWEINGPESVLVNTYQWIIRNWETVKIQWSQNPTMLYNKMEFEPFQSLVPKAIRGQYSGFVRTLFQQMRDVLGTFDTAQIIKLLPFAAAPPKQSRMQFSSFTVNVRGSGMRILVRGNSPVFNYNKATKRLTVLGKDAGTLTEDPDEGTAGVESAVLRGFLILGKEDKRYGPALSINELSNLAKGEKANVLIGQGDVVLVMKRKRDSSILTDSQTATKRIRMAIN

SEQ ID NO: 29 - protein, 757 aa, Influenza A virus
MDVNPTLLFLKVPAQNAISTTFPYTGDPPYSHGTGTGYTMDTVNRTHQYSEKGRWTTNTETGAPQLNPIDGPLPEDNEPSGYAQTDCVLEAMAFLEESHPGIFENSCIETMEVVQQTRVDKLTQGRQTYDWTLNRNQPAATALANTIEVFRSNGLTANESGRLIDFLKDVMESMKKEEMGITTHFQRKRRVRDNMTKKMITQRTIGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYMTRNQPEWFRNVLSIAPIMFSNKMARLGKGYMFESKSMKLRTQIPAEMLASIDLKYFNDSTRKKIEKIRPLLIEGTASLSPGMMMGMFNMLSTVLGVSILNLGQKRYTKTTYWWDGLQSSDDFALIVNAPNHEGIQAGVDRFYRTCKLLGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFEIKKLWEQTRSKAGLLVSDGGPNLYNIRNLHIPEVCLKWELMDEDYQGRLCNPLNPFVSHKEIESMNNAVMMPAHGPAKNMEYDAVATTHSWIPKRNRSILNTSQRGVLEDEQMYQRCCNLFEKFFPSSSYRRPVGISSMVEAMVSRARIDARIDFESGRIKKEEFTEIMKICSTIEELRRQK

SEQ ID NO: 30 - protein, 716 aa, Influenza A virus
MEDFVRQCFNPMIVELAEKTMKEYGEDLKIETNKFAAICTHLEVCFMYSDFHFINEQGESIIVELGDPNALLKHRFEIIEGRDRTMAWTVVNSICNTTGAEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRKLADQSLPPNFSSLENFRAYVDGFEPNGYIEGKLSQMSKEVNARIEPFLKTTPRPLRLPNGPPCSQRSKFLLMDALKLSIEDPSHEGEGIPLYDAIKCMRTFFGWKEPNVVKPHEKGINPNYLLSWKQVLAELQDIENEEKIPKTKNMKKTSQLKWALGENMAPEKVDFDDCKDVGDLKQYDSDEPELRSLASWIQNEFNKACELTDSSWIELDEIGEDVAPIEHIASMRRNYFTSEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLIRSAIGQVSRPMFLYVRTNGTSKIKMKWGMEMRRCLLQSLQQIESMIEAESSVKEKDMTKEFFENKSETWPIGESPKGVEESSIGKVCRTLLAKSVFNSLYASPQLEGFSAESRKLLLIVQALRDNLEPGTFDLGGLYEAIEECLINDPWVLLNASWFNSFLTHALS

SEQ ID NO: 31 - protein, 92 aa, Human immunodeficiency virus [sequence omitted]

SEQ ID NO: 32 - protein, 995 aa, Human immunodeficiency virus
LQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 33 - protein, 192 aa, Human immunodeficiency virus
MENRWQVMIVWQVDRMRIRTWKSLVKHHMYVSGKARGWFYRHHYESPHPRISSEVHIPLGDARLVITTYWGLHTGERDWHLGQGVSIEWRKKRYSTQVDPELADQLIHLYYFDCFSDSAIRKALLGHIVSPRCEYQAGHNKVGSLQYLALAALITPKKIKPPLPSVTKLTEDRWNKPQKTKGHRGSHTMNGH

SEQ ID NO: 34 - protein, 856 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 35 - protein, 116 aa, Human immunodeficiency virus
MAGRSGDSDEELIRTVRLIKLLYQSNPPPNPEGTRQARRNRRRRWRERQRQIHSISERILGTYLGRSAEPVPLQLPPLERLTLDCNEDCGTSGTQGVGSPQILVESPTVLESGTKE

SEQ ID NO: 36 - protein, 389 aa, Hepatitis B virus
MGTNLSVPNPLGFFPDHQLDPAFKANSENPDWDLNPHKDNWPDAHKVGVGAFGPGFTPPHGGLLGWSPQAQGILTSVPAAPPPASTNRQSGRQPTPLSPPLRDTHPQAMQWNSTTFHQTLQDPRVRALYFPAGGSSSGTVSPAQNTVSAISSILSKTGDPVPNMENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTCTTPAQGTSMFPSCCCTKPMDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 37 - protein, 780 aa, Hepatitis B virus
MPLSYQHFRKLLLLDDEAGPLEEELPRLADEGLNHRVAEDLNLGNPNVSIPWTHKVGNFTGLYSSTVPVFNPEWQTPSFPDIHLQEDIVDRCKQFVGPLTVNENRRLKLIMPARFYPNVTKYLPLDKGIKPYYPEHVVNHYFQTRHYLHTLWKAGILYKRESTHSASFCGSPYSWEQDLQHGRLVFQTSKRHGDKSFCPQSPGILPRSSVGPCIQSQLRKSRLGPQPTQGQLAGRPQGGSGSIRARIHPSPWGTVGVEPSGSGHTHICASSSSSCLHQSAVRTAAYSPISTSKGHSSSGHAVELHHFPPNSSRSQSQGSVLSCWWLQFRNSKPCSEYCLSHIVNLIEDWGPCAEHGEHRIRTPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQDLHNSCSRNLYVSLMLLYKTYGWKLHLYSHPIILGFRKIPMGVGLSPFLLAQFTSAICSVVRRAFPHCLAFSYMDDVVLGAKSVQHLESLYAAVTNFLLSLGIHLNPNKTKRWGYSLNFMGYVIGSWGTWPQDHIVQNFKLCFRKLPVNRPIDWKVCQRIVGLLGFAAPFTQCGYPALMPLYACIQAKQAFTFSPTYKAFLSKQYMTLYPVARQRPGLCQVFADATPTGWGLAIGHQRMRGTFVSPLPIHTAELLAACFARSRSGANLIGTDNSVVLSRKYTSFPWLLGCAANWILRGTSFVYV

SEQ ID NO: 38 - protein, 399 aa, Hepatitis B virus
  misc_feature (34)..(34): Xaa can be any naturally occurring amino acid
MGLSWTVPLEWGKNHSTTNPLGFFPDHQLDPAFXANTRNPDWDHNPNKDHWTEANKVGVGAFGPGFTPPHGGLLGWSPQAQGMLTTLPADPPPASTNRQSGRQPTPITPPLRDTHPQAMQWNSTTFHQALQDPRVRGLYFPAGGSSSGTVNPVPTTXSLISSIFSRIGDXAXXMESITSGFLGPLLVLQAGFFLLTKILTIPQSLDSWWTSLSFLGGAPVCLGQNSQSPTSNHSPTSCPPICPGYRWMCLRRFIIFLFILLLCLIFLLVLLDYQGMLPVCPLIPGSSTTSTGPXRTCTTLAQGTSMFPSCCCSKPSDGNCTCIPIPSSWAFGKFLWEWASARFSWLSLLVPFVQWFAGLSPTVWLSXIWMMWYWGPXLNNILSPFIPLLPIFFCLWVYI

(19) United States
(12) Patent Application Publication
(10) Pub. No.: US 2009/0208450 A1
(43) Pub. Date: Aug. 20, 2009

(54) METHOD FOR ENHANCING THE EFFICACY OF ANTIGEN SPECIFIC TUMOR IMMUNOTHERAPY

(76) Inventor: Xiao-Feng Yang, Huntingdon Valley, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 12/438,820

(22) PCT Filed: Aug. 24, 2007
(86) PCT No.: PCT/US07/76717

§ 371 Date: Feb. 25, 2009

Related U.S. Application Data
(60) Provisional application No. 60/823,776, filed on Aug. 29, 2006.

(51) Int. Cl. A61K 38/20; C12Q 1/02; A61K 39/00; A61K 38/21

(52) U.S. Cl. 424/85.2; 435/29; 424/177.1; 424/85.7; 424/85.5

(57) ABSTRACT

The invention provides a method for the improved processing efficiency of T cell tumor antigen epitopes using bioinformatic means. The proteolytic sites in the generation of 47 experimentally identified HLA-A2.1-restricted immunodominant tumor antigen epitopes was compared to those of 52 documented HLA-A2.1-restricted immunodominant viral antigen epitopes. The amino acid frequencies in the C-terminal cleavage sites of the tumor antigen epitopes, as well as several positions within the 10 amino acid (aa) flanking regions, were significantly different from those of the viral antigen epitopes. These two groups of epitopes may be cleaved by distinct sets of proteasomes and peptidases or similar enzymes with lower efficiencies for tumor epitopes, targeted activation of the immunoproteasomes and peptidases can be achieved that mediate the cleavage of viral epitopes in order to more effectively generate tumor antigen epitopes thus enhancing antigen-specific tumor immunotherapy.

### CROSS-REFERENCE TO RELATED APPLICATIONS

This application claims benefit of U.S. Provisional Patent Application No. 60/823,776, filed Aug. 29, 2006.

### STATEMENT REGARDING FEDERALLY SPONSORED RESEARCH OR DEVELOPMENT

This research was supported in part by U.S. Government funds (National Institutes of Health grant number AI054514), and the U.S. Government may therefore have certain rights in the invention.

### BACKGROUND OF THE INVENTION

1. Field of Invention

This invention relates to a method for the improved processing efficiency of T cell tumor antigen epitopes using bioinformatic means. The proteolytic sites in the generation of 47 experimentally identified HLA-A2.1-restricted immunodominant tumor antigen epitopes was compared to those of 52 documented HLA-A2.1-restricted immunodominant viral antigen epitopes. The amino acid frequencies in the C-terminal cleavage sites of the tumor antigen epitopes, as well as several positions within the 10 amino acid (aa) flanking regions, were significantly different from those of the viral antigen epitopes. In the 9 amino acid epitope region, frequencies differed somewhat in the secondary-anchored amino acid residues on E3 (the third aa of the epitope), E4, E6, E7, and E8; however, frequencies in the primary-anchored positions, on E2 and E9, for binding in the HLA-A2.1 groove remained nearly identical. The most frequently occurring amino acid pairs in both N-terminal and C-terminal cleavage sites in the generation of tumor antigen epitopes were different from those of the viral antigen epitopes. These two groups of epitopes may be cleaved by distinct sets of proteasomes and peptidases or similar enzymes with lower efficiencies for tumor epitopes, targeted activation of the immunoproteasomes and peptidases can be achieved that mediate the cleavage of viral epitopes in order to more effectively generate tumor antigen epitopes thus enhancing antigen-specific tumor immunotherapy.

2. Description of Related Art

Vaccines capable of eliciting T cell immune responses have been successfully developed for prevention of 26 viral and bacterial infectious diseases (1). In contrast, despite significant progress (2), effective vaccines for most types of tumor are still lacking (3). Since most tumor antigens reported are nonmutated self-antigens (2), peripheral T cell repertoire may be tolerized to self-antigens via thymic negative selection of autoreactive T cells but reacted to viral (foreign) antigens. This model of self-tolerance via thymic selection is often considered as a mechanism of underlying the efficiency differences between the vaccines against viral infections and that against tumors (4). However, self-tolerance, based on the avidity of T cells for self-MHC (major histocompatibility complex)/self-peptide complexes in the thymic selection process, is far from absolute (4). T cells with low avidity for ubiquitously expressed self-antigens or low level expressed self-antigens can escape clonal deletion in thymus and enter the periphery (4). Thus, thymic tolerance is one of the important factors but not the only factor in determining T cell immune responses to tumors and viral infection. T cell responses are also regulated by the process of antigen processing. Improving antigen processing of tumor antigens has been proposed to be a very important direction in development of novel vaccination strategies against tumors (5). Recent reports demonstrated that interferon (IFN)-gamma, which is secreted in large amounts during viral infections (6), alters enzymatic processing and proteolytic specificities in generation of T cell antigen epitopes via induction of immunoproteasomes (7) and novel aminopeptides (8). In other words, these results suggested that proteasomes and other enzymes in generation of T cell antigen epitopes versus viral antigen epitopes could be different due to differential expression of IFN-gamma during viral infections and tumor growth (7). Notwithstanding, the comprehensive features of proteolytic cleavage sites involved in differential generation of tumor antigen epitopes and viral antigen epitopes remain unknown.

For generation of MHC class I-restricted antigen epitopes, several requirements have been identified, including the following: 1) cleavage sites and favorable flanking sequence around cleavage sites can be effectively recognized by ubiquitin-proteasome complex(9,10)—although nonproteasomal mechanisms also seem to be involved in antigen processing(11,12); 2) transported antigen epitopes have high affinity for binding to transporter associated protein (TAP)(13,14) and for being transported to MHC class I complex (15); and 3) antigen epitopes have high affinity for binding and stabilizing HLA (human leukocyte antigen, human MHC) class I complex on the cell surface (16) (also see Schreuder G M, Hurley C K, Marsh S G, Lau M, Fernandez-Vina M A, Noreen H J, Setterholm M, Maiers M. HLA dictionary 2004: summary of HLA-A, -B, -C, -DRB1/3/4/5, -DQB1 alleles and their association with serologically defined HLA-A, -B, -C, -DR, and -DQ antigens. Hum Immunol. 2005 February; 66(2):170-210). It is well accepted that a small subset of peptides generated by proteasomes and processing peptidases, transported by TAP, loaded on MHC class I (17) are potent in elicitation of T cell immune responses and become immunodominant epitopes(18,19), which are desirable for development of novel immunotherapy.

Recently developed serological analyses of tumor antigens by recombinant expression cDNA cloning (SEREX)(20,21) have led to the identification of a large number of tumor antigens (22), which hold great promise as targets for novel antigen-specific tumor immunotherapy (23,24). Previously, the inventor identified broadly immunogenic SEREX tumor antigens (25), CML66L (26,27) and CML28 (28), with which specific high-titer IgG antibody responses were associated in the remission of chronic myelogenous leukemia (CML)(25,26,28). Recently, the inventor's findings indicated that the overexpression of CML66L in tumor cells, mediated by alternative splicing, is the mechanism of the immunogenicity of this antigen, suggesting that overexpression of SEREX-identified tumor antigens by vaccination could generate anti-tumor immune responses (29). Immunization using dominant antigenic peptides has been most effective in patients with tumors (30) and has generated surprisingly high levels of circulating T cells directed against tumor antigens with a therapeutic outcome (31). Thus, immunodominant epitopes capable of eliciting remarkable CD8+ T cell responses would contribute decisively to the improvement of peptide-based immunization protocols for patients with tumors (32). However, due to an incomplete understanding of the mechanism underlying the generation of immunodominant (measurable T cell reactive) epitopes, as well as technical limitations, the identification of immunodominant T cell antigen epitopes from SEREX antigens has been accomplished at a slow pace; nonetheless, a few SEREX antigens (i.e., MAGE-1, tyrosinase, NY-ESO-1, coactosin-like protein and CML66) are reported to have the ability to elicit both cellular and humoral immune responses to tumor cells (29,33,34). To facilitate the identification of T cell reactive epitopes encoded by a large number of the SEREX antigens, two important questions must be addressed: 1) whether the structures around the cleavage sites generating the T cell reactive tumor antigen epitopes are different from those of identified immunodominant viral antigen epitopes; and 2) if bioinformatic features of the cleavage sites generating the dominant tumor antigen epitopes can be extracted using a statistical approach, whether the processing efficiency of immunodominant tumor epitopes can be improved thereby in the future.

The invention provides that proteolytic cleavage sites generating the identified immunodominant tumor antigen epitopes are statistically different from those generating documented immunodominant viral epitopes. The inventor focused on the statistical analysis of HLA-A2.1-restricted tumor antigen nonapeptide epitopes and viral antigen nonapeptide epitopes that were previously identified to be T cell reactive through the experimental approaches of others (18,19) (also, see the web database: www.cancerimmunity.org/peptide database/Tcellepitopes.htm). The purpose in establishment of the public database(s) of T cell antigen epitopes is for database mining to reveal novel information. The statistical approach that was applied has the advantage of revealing important information on structural features through biochemical analysis of individual antigen epitopes, as we demonstrated previously (35). Of note, in contrast to the biochemical analyses with epitope peptides digestible by proteasomes or peptidases, the inventor focused on analyzing the experimentally identified HLA-A2.1 restricted T cell reactive epitopes, which are desirable for future development of antigen specific immunotherapy (32, 35). Defining the features shared by experimentally identified tumor antigen epitope cleavage sites in a statistical approach would be a very important key to understanding the generation of tumor antigen epitopes versus that of the viral antigen epitopes. Experimentally identified, HLA-A2.1-restricted T cell reactive tumor antigen epitopes share structural features around the cleavage sites, but that these structural features were not identical to those used in the generation of viral antigen epitopes. New discoveries through the panoramic analysis, in return, have justified this bioinformatic approach. With such knowledge, the inventor has the ability to make processing of tumor antigen epitopes more efficiently, and improve tumor immunotherapy.

All references cited herein are incorporated herein by reference in their entireties.

### BRIEF SUMMARY OF THE INVENTION

The invention provides a method for characterizing a tumor antigen to serve as the antigen for the generation of tumor specific vaccines capable of eliciting a T cell immune response comprising: (a) identifying HLA restricted T cell reactive epitopes; (b) identifying proteolytic cleavage sites of the HLA restricted T cell reactive epitopes; (c) statistically analyzing the identified immunodominant tumor antigen epitopes to determine whether they are statistically different from those of known documented immunodominant viral epitopes; wherein a tumor antigen statistically different from those of known documented immunodominant viral epitopes can serve as the antigen for the generation of tumor specific vaccines capable of eliciting T cell immune response.

The invention provides a method of selecting a tumor antigen to serve as the antigen for the generation of tumor specific vaccines capable of eliciting a T cell immune response comprising: (a) identifying HLA restricted T cell reactive epitopes; (b) identifying proteolytic cleavage sites of the HLA restricted T cell reactive epitopes; (c) statistically analyzing the identified immunodominant tumor antigen epitopes to determine whether they are statistically different from those of known documented immunodominant viral epitopes; (d) selecting the identified immunodominant tumor antigen epitopes which are statistically different from those of known documented immunodominant viral epitopes; wherein a tumor antigen statistically different from those of known documented immunodominant viral epitopes can serve as the antigen for the generation of tumor specific vaccines capable of eliciting T cell immune response.

The invention provides a method for the generation of tumor specific vaccines capable of eliciting a T cell immune response comprising: (a) identifying HLA restricted T cell reactive epitopes; (b) identifying proteolytic cleavage sites of the HLA restricted T cell reactive epitopes; (c) statistically analyzing the identified immunodominant tumor antigen epitopes to determine whether they are statistically different from those of known documented immunodominant viral epitopes; (d) selecting the identified immunodominant tumor antigen epitopes which are statistically different from those of known documented immunodominant viral epitopes; (e) making a pharmaceutical composition comprising one or more of the identified immunodominant tumor antigen epitopes as a tumor specific vaccine capable of eliciting a T cell immune response; (f) administering the pharmaceutical composition; thereby eliciting a T cell immune response. The invention further provides the method, wherein the pharmaceutical composition further comprises one or more members selected from the group consisting of a cytokine, a chemotherapeutic agent, a chemokine, and an adjuvant. The invention further provides the method, wherein the cytokine is selected from the group consisting of a tumor necrosis factor, an interleukin, a lymphokine, granulocyte colony-stimulating factor (G-CSF), a granulocyte macrophage colony-stimulating factor (GM-CSF), a macrophage colony-stimulating factor (M-CSF), monocyte chemoattractant protein 1 (CP1), macrophage inflammatory protein MIP1α, macrophage inflammatory protein MIP1β, IL-1, IL-2, IL-3, IL4, IL-5, IL-6, IL-7, IL-8, IL-9, IL-10, IL-11, IL-12, IL-13, IL-14, IL-15, IL-16, IL-17, IL-18, TNF-α, IFN-α, IFN-γ, and IL-20 (MDA-7).

The invention provides a method of treatment of cancer by administration of tumor specific vaccines capable of eliciting a T cell immune response comprising: (a) identifying HLA restricted T cell reactive epitopes; (b) identifying proteolytic cleavage sites of the HLA restricted T cell reactive epitopes; (c) statistically analyzing the identified immunodominant tumor antigen epitopes to determine whether they are statistically different from those of known documented immunodominant viral epitopes; (d) selecting the identified immunodominant tumor antigen epitopes which are statistically different from those of known documented immunodominant viral epitopes; (e) making a pharmaceutical composition comprising one or more of the identified immunodominant tumor antigen epitopes as a tumor specific vaccine capable of eliciting a T cell immune response; (f) administering the pharmaceutical composition to a patient; thereby treating the patient. The invention further provides the method, wherein the composition comprising the tumor specific vaccine capable of eliciting T cell immune response further comprises one or more members selected from the group consisting of a cytokine, a chemotherapeutic agent, a chemokine, and an adjuvant. The invention further provides the method, wherein the cytokine is selected from the group consisting of a tumor necrosis factor, an interleukin, a lymphokine, granulocyte colony-stimulating factor (G-CSF), a granulocyte macrophage colony-stimulating factor (GM-CSF), a macrophage colony-stimulating factor (M-CSF), monocyte chemoattractant protein 1 (CP1), macrophage inflammatory protein MIP1α, macrophage inflammatory protein MIP1, IL-1, IL-2, IL-3, IL4, IL-5, IL-6, IL-7, IL-8, IL-9, IL-10, IL-11, IL-12, IL-13, IL-14, IL-15, IL-16, IL-17, IL-18, TNF-α, IFN-α, IFN-γ, and IL-20 (MDA-7).

The invention provides an isolated tumor specific vaccine capable of eliciting a T cell immune response identified by the method comprising: (a) identifying HLA restricted T cell reactive epitopes; (b) identifying proteolytic cleavage sites of the HLA restricted T cell reactive epitopes; (c) statistically analyzing the identified immunodominant tumor antigen epitopes to determine whether they are statistically different from those of known documented immunodominant viral epitopes; (d) selecting the identified immunodominant tumor antigen epitopes which are statistically different from those of known documented immunodominant viral epitopes; (e) making a pharmaceutical composition comprising one or more of the identified immunodominant tumor antigen epitopes as a tumor specific vaccine capable of eliciting a T cell immune response; wherein a tumor antigen statistically different from those of known documented immunodominant viral epitopes can serve as the antigen for the generation of tumor specific vaccines capable of eliciting T cell immune response. The invention further provides a tumor specific vaccine capable of eliciting a T cell immune response, comprising an epitope as set forth in FIG. 4.

The invention provides a kit comprising the tumor specific vaccine capable of eliciting a T cell immune response, further comprising an adjuvant, and a pharmaceutically acceptable carrier. The invention also provides the kit further comprising one or more members selected from the group consisting of a cytokine, a chemotherapeutic agent, a chemokine, and an adjuvant. The invention also provides the kit, wherein the cytokine is selected from the group consisting of a tumor necrosis factor, an interleukin, a lymphokine, granulocyte colony-stimulating factor (G-CSF), a granulocyte macrophage colony-stimulating factor (GM-CSF), a macrophage colony-stimulating factor (M-CSF), monocyte chemoattractant protein 1 (CP1), macrophage inflammatory protein MIP1α, macrophage inflammatory protein MIP1β, IL-1, IL-2, IL-3, IL4, IL-5, IL-6, IL-7, IL-8, IL-9, IL-10, IL-11, IL-12, IL-13, IL-14, IL-15, IL-16, IL-17, IL-18, TNF-α, IFN-α, IFN-γ, and IL-20 (MDA-7).

### BRIEF DESCRIPTION OF SEVERAL VIEWS OF THE DRAWINGS

The invention will be described in conjunction with the following drawings in which like reference numerals designate like elements and wherein:

FIG. 1. Position nomenclature of the epitopes and the flanking regions. FIG. 1A. Schechter and Berger's enzymatic cleavage nomenclature is used in reference to the amino acid positions in the N-terminal and C-terminal flanking regions, relative to their respective cleavage sites. FIG. 1B. Two cleavage sites are required during the final step of antigen epitope generation. Since there is an overlap between the C-terminal flanking region of the epitope's N-terminal cleavage site and the N-terminal flanking region of the epitope's C-terminal cleavage site, in order to avoid potential confusion, the amino acids in the epitope (E1 to E9), the N-terminal flanking region (N10-N1), and the C-terminal flanking region (C1-C10) are uniquely defined.

FIG. 2. Comparison of the amino acid pairs located in the N-terminal cleavage sites (Pn1-Pn1′) and the C-terminal cleavage sites (Pc1-Pc1′) in the tumor antigen and viral antigen epitopes. FIG. 2A. Schematic representation of the notation for the two overlapping sets of amino acid pairs. For the computation of probability mass function, the overlapped amino acid pairs in set 1 (tumor epitopes) are assigned as r1, the corresponding remainders (nonoverlapping) in the same set are assigned as k1-r1. Similarly, the overlapped amino acid pairs (nonoverlapping) in set 2 (viral epitopes) are assigned as r2, the corresponding remainders in the same set are assigned as k2-r2. FIG. 2B. Schematic representation of the percentages of identical amino acid pairs versus the different amino acid pairs in the tumor antigen and viral antigen epitopes. Since identical amino acid pairs can be present more than once, percentages for the two groups are slightly different. Note that there are twenty amino acids; thus, theoretically, there are 400 possible amino acid pairs. FIG. 2C. The frequently occurring amino acid pairs (i.e., those occurring more than once) located in the N-terminal cleavage sites (Pn1-Pn1′) and the C-terminal cleavage sites (Pc1-Pc1′) in the tumor antigen and viral antigen epitopes. Percentages of the amino acid pairs in the tumor epitope or the viral epitope group are presented as frequencies. The physical characteristics of the amino acids are indicated through the use of different font formats: acidic, underline; basic, italic; hydrophobic, bold; and neutral, grey. FIG. 2D. The surface plot of probability mass function. The ordinate indicates the number of overlapped amino acid pairs (r2) in the viral epitopes, the abscissa indicates the number of overlapped amino acid pairs (r1) in the tumor epitopes, and the Z-axis indicates the probability mass function. The probability mass function demonstrates the distribution of the probability for any given overlaps of amino acid pairs in the N-terminal cleavage sites and C-terminal sites in two sets. The curve line indicates the probability for the 95% of all the random overlaps of amino acid pairs (95% confidential interval). FIG. 2E. The contour plot of probability mass function. The events sharing the probability are linked by a contour curve. The ordinate indicates the number of overlapped amino acid pairs (r1) in the tumor epitopes, and the abscissa indicates the number of overlapped amino acid pairs (r2) in the viral epitopes. A loop with the probability of 0.005 indicates the probability for the 95% of all the random overlaps of amino acid pairs (95% confidential interval), which corresponds to the curve line presented in FIG. 2D. The probabilities to have the amino acid pairs overlapped at the N-terminal sites and C-terminal sites in two sets of epitopes (FIG. 2B) are marked in the plot by a triangle and a star, respectively.

FIG. 3. Comparison of proteasome cleavage scores, and the binding potential of TAP and HLA-A2.1 in the tumor versus viral epitopes. FIG. 3A. Schematic representation of the differences in predicted proteasome cleavage scores of the tumor and viral epitopes. Prediction of proteasome cleavage scores of the epitope-containing antigen sequences was performed by using the algorithms MHC-pathway constitutive proteasomes, MHC-Pathway immunoproteasome, NetChop3.0, and MAPPP. The mean±1.96 SE of the proteasome cleavage scores of the antigen sequences was calculated for a 95% confidence interval (95% CI). The resulting lack of overlap in the 95% CI indicates that there is a difference in proteasome cleavage scores for the tumor versus viral epitopes. FIG. 3B. Statistical comparison of proteasome cleavage scores, TAP binding potential, and HLA-A2.1 binding potential of the tumor versus viral epitopes, predicted through commonly used algorithms. The BIMS value of antigen epitopes was transformed by Ln function before the statistical analyses.

FIG. 4. Antigens. FIG. 4A. Tumor Antigen epitopes were derived from tumor antigens, which included representatives from the four tumor antigen groups characterized thus far: the group of differentiation antigens, including tyrosinase, gp100; the group of amplified/oncogenic antigens, including HER-2/neu, WT1; the group of mutational antigens, including p53; and the group of cancer-testis antigens, including MAGE and NY-ESO-1. FIG. 4B. Immunodominant 52 HLA-A2.1-restricted viral antigen epitopes, along with nine amino acid epitope residues, and the ten amino acids in the N-terminal and C-terminal flanking regions.

FIG. 5. Usage of amino acid residues in the tumor antigen epitopes was less diversified than that of the viral epitopes. In positions E2, E3, and E9, amino acid preference was conserved in the two groups of epitopes. The results of this conservation in the primary HLA-A anchor residues, on E2 and E9, and the secondary anchor residue, on E3, corresponded to prior reports emphasizing the dominant structural requirement for HLA-A2.1 binding, which corresponded to the previous findings (e.g., Leu or Met at position E2, and Val, Leu, or Ile at position E9 of the epitope regions).

### DETAILED DESCRIPTION OF THE INVENTION

The preference of amino acids in the flanking positions of tumor antigen epitopes was different from that of the viral antigen epitopes. In order to determine whether cleavage sites in the generation of tumor antigen epitopes were different from those of viral epitopes, we analyzed all of the 47 HLA-A2.1-restricted immunodominant tumor antigen epitopes experimentally identified so far (18,19) and identified immunodominant 52 HLA-A2.1-restricted viral antigen epitopes, along with nine amino acid epitope residues, and the ten amino acids in the N-terminal and C-terminal flanking regions. These 47 epitopes were derived from 24 tumor antigens (see Table I), which included representatives from the four tumor antigen groups characterized thus far: the group of differentiation antigens, including tyrosinase, gp100; the group of amplified/oncogenic antigens, including HER-2/neu, WT1; the group of mutational antigens, including p53; and the group of cancer-testis antigens, including MAGE and NY-ESO-1. This collection allowed us to analyze the common structural features of the cleavage sites and the epitopes shared by various tumor antigens. For the purpose of comparison, we also collected 52 HLA-A2.1-restricted viral antigen epitopes encoded by HIV, HBV, HCV, and influenza A virus, as the reference epitopes. These viral antigen epitopes were suitable for comparison, due to the inclusion of both DNA and RNA viruses, which were categorized into several virus families, including the retroviridae (HIV), the hepadnaviridae (HBV), the flaviviridae (HCV), and the orthomyxoviridae (influenza virus A)(48).

Initially the mean and variance of the processing probabilities of tumor epitopes and viral epitopes were calculated by using the MAPPP algorithm. Based on these results, an estimation of sample size, in comparing the means of the tumor epitope group and the viral epitope group, was calculated according to previously published statistical methods(49). The results indicated that, in each group, 33 epitopes or more must be included in order to obtain 80% power (not shown); thus, the number of epitopes in the tumor epitope group (47 epitopes) and the viral epitope group (52 epitopes) exceeded the calculated power requirement (49). Of note, the statistical estimation of sample size suggested that the conclusion achieved in this study, with more than the sufficient sample size to gain the >80% high power levels is statistically significant, but not biased (49).

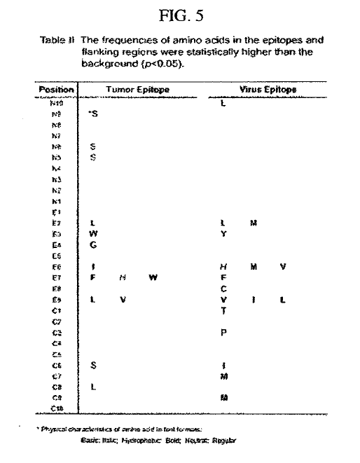

The statistical differences in the frequency of each amino acid in 29 positions of the tumor antigen epitopes and the viral antigen epitopes were analyzed, and the flanking regions in comparison to the general occurrence frequencies (47). Since both groups of antigen epitopes were compared against the same control amino acid occurrence frequencies (47), the results from these two groups were comparable. The amino acid frequencies that are statistically higher than the background are listed in FIG. 5. Several findings were reported: 1) at 17 out of the 29 positions, amino acid distributions in the tumor antigen and viral antigen epitopes deviated significantly from the background (FIG. 5) (p<0.05); 2) in the flanking region positions, on N9, N6, N5, and C8, only the tumor antigen—and not the viral antigen—epitopes deviated from the background; and (3) in the flanking region positions, on Nb1, C1, C3, C7, and C9, only the viral antigen—and not the tumor antigen—epitopes deviated from the background. Previous studies showed some amino acid preferences in the N-termini (Pn1′) and the C-termini (Pc1′) of the proteasome cleaved epitopes. These studies also showed that the C-terminal cleaved position (Pc1′) prefers K, R, A, and S, but does not favor F, D, and E(17). In contrast, data did not find statistically significant differences in the amino acid occurrence frequencies in position Pn1′ of either the tumor antigen or viral antigen epitopes (p>0.05). In addition, data on viral antigen epitopes showed that this C-terminal cleavage position (Pc1′) favored T. analyses indicated that both the N-terminal and the C-terminal cleavage sites of the tumor antigen epitopes were different from those of the viral antigen epitopes.

Cumulatively, these results demonstrate the following. First, the epitope flanking regions of tumor and viral epitopes have amino acid preferences that are statistically different from the general amino acid frequency background. The following reports support design in using general amino acid frequency as a background: (1) In contrast to bacteria, human viruses do not have their own protein translation machinery, and need to use human cell protein translation machinery for synthesis of viral proteins(48); (2) Since viruses can be efficiently replicated in human cells, human cell protein translation system must be capable of efficient translation of viral proteins(48); (3) Codon usage and amino acid frequencies of human proteins and viral proteins synthesized in human cells are generally determined by the expression levels of tRNAs with appropriate anticodons in human cells(50,51). Second, differences in amino acid preferences on positions in the flanking regions can be observed between the viral and tumor epitopes. Although the epitope positions were considered in the flanking regions for both the N-terminal and C-terminal enzyme cleavage sites, it is possible that amino acid restriction in the epitope positions for HLA (52-54) and TAP binding(13,14) may override enzymatic cleavage influences at both ends. Third, and most important, there are significant differences between these two groups of epitopes in positions N1 (Pn1), E1 (Pn1′), and C1 (Pc1′), suggesting that there are differences in the proteolytic enzymes involved in the generation of these two groups of epitopes.

Usage of amino acid residues in the tumor antigen epitopes was less diversified than that of the viral epitopes. As shown in FIG. 5, in positions E2, E3, and E9, amino acid preference was conserved in the two groups of epitopes. The results of this conservation in the primary HLA-A anchor residues, on E2 and E9, and the secondary anchor residue, on E3, corresponded to prior reports emphasizing the dominant structural requirement for HLA-A2.1 binding, which corresponded to the previous findings (e.g., Leu or Met at position E2, and Val, Leu, or Ile at position E9 of the epitope regions)(52-54). It should be noted that an auxiliary anchor at E3 usually fine-tunes peptide recognition (55,56). In study, the high restriction in both tumor antigen and viral antigen epitopes served as an appropriate positive control for the quality of analyses. The significant differences between the two groups of epitopes in positions E4, E6, E7, and E8 suggest that HLA-A2.1 binding (52-54) and TAP binding(13,14) do not have high restriction in these positions. The results also suggest that the differences in these positions between the two groups of epitopes may reflect variances in enzyme recognition in the flanking regions, since the epitope region serves as the C-terminal flanking region for N-terminal cleavage, as does the N-terminal flanking region for C-terminal cleavage. Future work will also need to examine whether the structural features in the auxiliary anchor positions contribute to lower binding avidity between interaction of MHC/self-tumor antigen peptides and T cell antigen receptor (TCR) and higher binding avidity between MHC/viral peptides and TCR (4).

There are no sequence structures for proteasome cleavage sites inside the epitopes, as defined by two hydrophobic residues at the E2 and E9 positions. This finding suggests that the epitope candidates having both the right HLA-A2.1 anchor residues on E2 and E9 and the internal cleavage sites should have been degraded, and there is no opportunity for these epitopes to be presented. Similarly, there are no sequence structures for HLA-A2.1 anchor residues and proteasome cleavage sites within the 10 amino acid residues in the epitope N-terminal and C-terminal flanking regions, suggesting that a special feature of the flanking regions is to enable the efficient processing of epitopes.

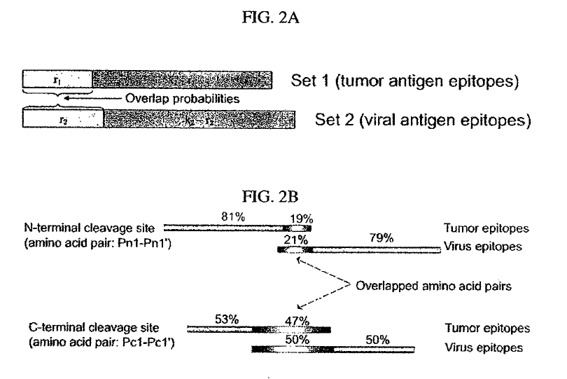

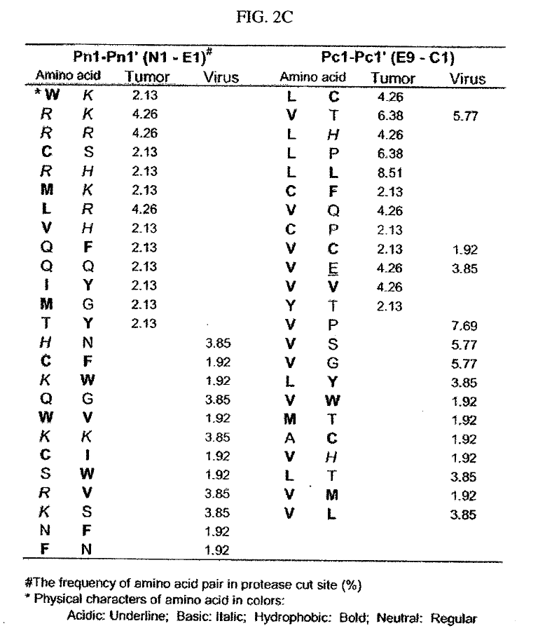

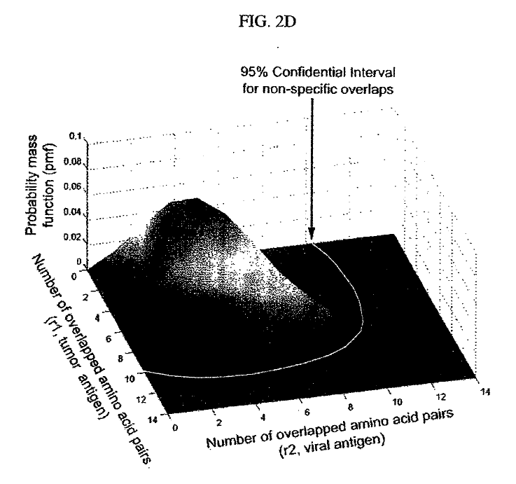

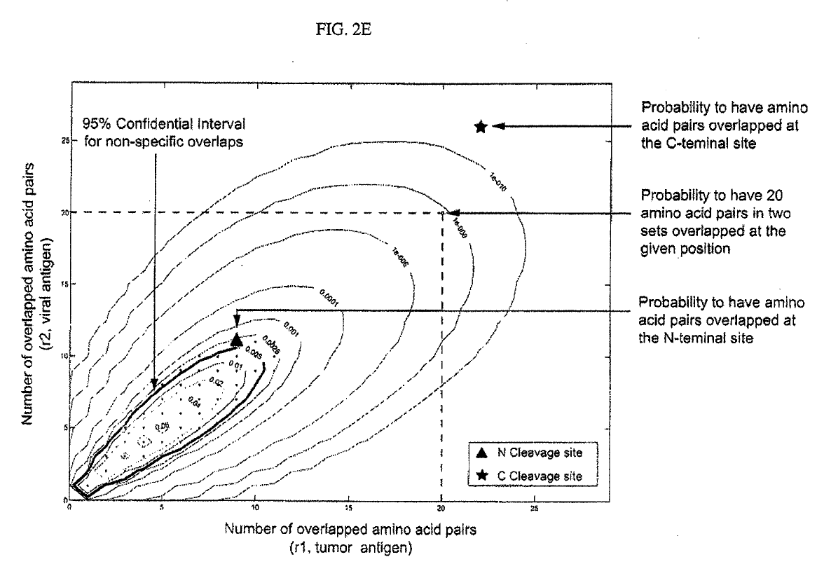

The amino acid pairs covering the cleavage sites in the generation of tumor antigen epitopes were different from those of the viral antigen epitopes. Protein sequences encode more structural and functional information than amino acid occurrence frequencies. In order to further explore this difference, we compared occurrence frequencies of the amino acid pairs(17) in the Pn1 (N1)-Pn1′ (E1) and the Pc1 (E9)-Pc1′ (C1) of the tumor and viral epitopes. These positions were selected to compare amino acid pairs because they are primary structural features for enzyme recognition and cleavage(40). The invention provides that if the proteasomes and peptidases that process the immunodominant tumor epitopes are the same as or similar to that processing the immunodominant viral epitopes, the amino acid pairs that are identical in these two groups of epitopes would be in high percentages in these positions. As shown in FIGS. 2B and 2C, the occurrence of amino acid pairs in N-terminal cleavage sites of the tumor epitopes was radically different from that of the viral antigen epitopes. In the N-terminal cleavage site (the Pn1-Pn1′ pair, depicted in FIG. 2B), out of 400 possible pairs, 81% of the total pairs in the tumor epitopes and 79% of the total pairs in the viral epitopes were different. In FIG. 2C, the most frequently occurring 13 pairs consisted of 34.0% of tumor epitopes. The most frequently occurring 12 pairs in the viral epitopes covered 32.7% of the epitope group. In addition, the frequency of pairs with the basic amino acid at the Pn1 position of the pairs was significantly increased, from 10.6% of the most frequently occurring tumor epitopes, to 17.3% of the most frequently occurring viral epitopes, suggesting that increased trypsin-like activity mediates the processing of viral epitopes (10). Moreover, the frequency of pairs with hydrophobic amino acid at the Pn1 position of the pairs was significantly decreased, from 17.0% of the most frequently occurring tumor epitopes, to 7.7% of the most frequently occurring viral epitopes, suggesting that an increase in chymotrypsin-like activities is responsible for tumor epitope processing (10). Finally, the frequency of pairs with the basic amino acid at the C-position of the pairs was significantly decreased, from 21.3% of the most frequently occurring tumor epitopes, to 3.9% of the most frequently occurring viral epitopes. Again, these results demonstrate that the N-terminal cleavages of both the tumor and viral epitopes are mediated by two different groups of enzymes.

Similarly, the occurrence of amino acid pairs in the C-terminal cleavage sites of the tumor antigen epitopes (the Pc1-Pc1′ pair) was different from that of the viral antigen epitopes. In FIG. 2B, 53% of the total pairs in the tumor epitopes did not share with 50% of the total pairs in the viral epitopes. The most frequently occurring 12 pairs covered 51.1% of tumor epitopes, and the most frequently occurring 14 pairs covered 51.9% of viral epitopes. In FIG. 2C, among those most frequently occurring pairs, three pairs were conserved between the tumor and viral epitope groups, comprising only 11.5% of the C-terminal cleavage sites. Moreover, differences in the physical features of amino acid pairs in the tumor epitopes versus the viral epitopes were less obvious in the C-terminal cleavage sites, in comparison with the N-terminal cleavage sites. Hydrophobic residues were present in most Pc1 positions of the most frequently occurring pairs, suggesting that chymotrypsin-like activity may be dominant in the processing of C-terminal cleavages of tumor and viral epitopes (10) in addition to the HLA-A2.1 binding preference at the position Pc1/E9 (52-54) and the TAP binding preference at this position (13,14). These results suggest that pairs in the C-terminal sites are less diversified than those in the N-terminal sites. findings show that the proteolytic enzymes generating C-terminal cleavage sites of tumor and viral epitopes are also different.

Furthermore, in order to determine whether the percentages of amino acid pair overlapped in the N-terminal site and C-terminal site in the set of 47 tumor antigen epitopes and the set of 52 viral epitopes are statistically significant, the computation for the probability of amino acid pairs overlapped in two steps was performed. First, the probability mass function (pmf) for all the random overlaps of amino acid pairs in two sets of epitopes was analyzed with the surface plot (FIG. 2D), which had the visual demonstration of the probability distribution of random overlaps of amino acid pairs in two sets of epitopes The results in FIG. 2D showed that the probability for the 95% of all the random overlaps of amino acid pairs (the “mountain area” in the surface plot) in the two sets of epitopes was ≧0.004; and the overlaps of amino acid pairs in two sets of epitopes with the probability <0.004 were not random. The results indicate that higher percentages of amino acid pairs overlapped in two sets, having the probability <0.004, reflected the shared specificities between the enzymes of cleaving tumor epitopes and the enzymes of cleaving viral epitopes to some extent. Second, the same analysis on the probability mass function was performed using the contour plot (FIG. 2E). The results showed that the amino acid pairs overlapped in the two sets of epitopes along the same probability contour line had the same probability. The 95% of all the random overlaps of amino acid pairs between tumor epitopes and viral epitopes was within (≧) the 0.004 probability contour loop, which was designated as 95% confidential interval (CI) loop. Once again, the overlaps of amino acid pairs in two sets of epitopes with the probability <0.004, outside of the 95% CI loop were not random. Interestingly, the probability for the overlapped amino acid pairs in the N-terminal cleavage sites (FIG. 2B) was 3.3×10-3; the probability for the overlapped amino acid pairs in the C-terminal sites (FIG. 2B) was 3.4×10-12. Therefore, both probabilities were smaller than 0.004. In biochemical terms, if no enzymatic specificity is shared in cleavage of the N-terminal sites or C-terminal sites for two sets (in totally random conditions), the probability to have amino acid pairs overlapped in these cleavage sites of two sets of epitopes should be ≧0.004 (FIG. 2E). As shown in FIG. 2E, since the probabilities to have amino acid pairs overlapped at the N-terminal site and at the C-terminal site were not random, the enzymatic specificities for cleaving the amino acid pairs were required to share about 20% to 50% amino acid pair overlaps (FIG. 2B) in two sets of epitopes. The enzyme(s) for cleaving the amino acid pairs in the C-terminal site (50% amino acid overlaps) shared more specificities than that in the N-terminal site (about 20% amino acid pair overlaps) (FIG. 2B). Since the probability for overlaps of amino acid pairs in the N-terminal sites in two sets was near the 95% confidential interval loop for random overlaps, the shared specificities for the N-terminal sites for two sets were minimal. If the percentages for overlaps of amino acid pairs in the N-terminal sites and C-terminal sites are 100% or close to 100%, the proteolytic enzymes mediating cleavage in these two sets of epitopes should be the same. Since the percentages for overlaps of amino acid pairs in the N-terminal sites and C-terminal sites were far less than 100%, the cleavages in both sites are mediated by two different groups of enzymes.

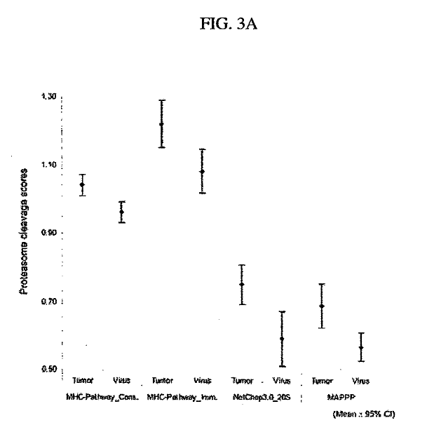

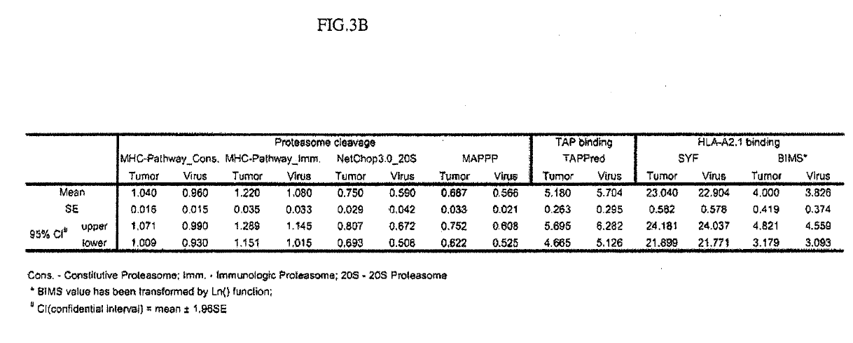

Statistically significant differences were found in the proteasome cleavage probability of tumor versus viral epitopes. The inventors further examined whether there are any differences between the tumor antigen and viral antigen epitopes by employing the commonly adopted algorithms for the prediction of processing probability by proteasome and immunoproteasome. The reason for choosing the following four algorithms, the MAPPP (www.mpiib-berlin.mpg.de/MAPPP/cleavage.html)(42), the MHC-Pathway constitutive proteasome (www.mhc-pathway.net/), the MHC-Pathway immunoproteasome, and the NetChop3.0 neural network predictor (www.cbs.dtu.dk/services/NetChop/)(44) rather than other algorithms, such as PAProC (www.paproc.de) (57), is that the former four algorithms allow quantitative prediction. Of note, experimental methods chosen for experiments are not due to their perfection. Likewise, the chosen algorithms may not have 100% prediction efficiency. For any given tumor antigens, these four algorithms may predict some common cleavage sites as well as the different sites. However, using the same sets of algorithms to analyze tumor epitopes and viral epitopes, the results from both sets of epitopes are statistically comparable; the common sites predicted by all four algorithms may reflect the common features of proteasome cleavage revealed from different angles. As shown in FIGS. 3A and B, predicted by the algorithm MAPPP, for example, the mean±1.96 standard error (SE) (95% confidence interval, CI) of the proteasome cleavage scores, for tumor antigen epitopes were between 0.62 and 0.75. In contrast, the 95% CI of the scores for viral antigen epitopes predicted with the same algorithm was between 0.53 and 0.61. To further consolidate this finding, similar analyses with three other algorithms for proteasome peptide cleavage, including the NetChop 3.0 and MHC-Pathway constitutive proteasome and MHC-Pathway immunoproteasomes were applied. As shown in FIGS. 3A and B, the results obtained were similar to that achieved with the algorithm MAPPP. Of note, the scores of both sets of antigen epitopes predicted with the algorithm MHC-Pathway immunoproteasome were higher than that predicted with the algorithm MHC-Pathway constitutive proteasome. With all four algorithms, there were no overlaps in the 95% CI of the predictive scores for these two groups of antigen epitopes, and a two-sided p<0.05, based on the Wilcoxon rank-sum test, suggested that tumor antigen epitopes are different from viral epitopes regarding their probability of being processed by proteasomes and immunoproteasomes (p<0.05). The results indicated that potential tumor antigen epitopes could not be processed efficiently. The lower probability that viral epitopes will be processed by these proteasomes suggests that the “threshold” for processing tumor antigen epitopes by proteasomes and immunoproteasomes is higher than that for viral antigen epitopes.

In FIG. 3B, the Wilcoxon rank-sum test comparison of the TAP-binding potential (predicted by the algorithm TAPPred)(15) of tumor antigen epitopes and viral antigen epitopes showed that the viral epitopes had a slightly higher potential than the tumor epitopes to bind to TAP for transfer into ER—even though there were no statistical differences (p>0.05). Furthermore, a comparison of the HLA-A2.1-binding potential of the tumor antigen and viral epitopes—predicted by the algorithms SYF [SYFPEITHI] (58) and BIMAS [BIMAS/NIH](54)—demonstrated a similarity in the two groups of epitopes, suggesting that HLA-A2.1 restriction had overcome the differences in amino acid occurrences and physical characteristics. These results correlated with the fact there are only one type of HLA-A2.1 and limited human TAP polymorphism (13,59) for binding of all the epitopes regardless of tumor antigens or viral antigens. Based on analyses of the experimentally identified 47 tumor antigen epitopes and 52 viral antigen epitopes, generation of a 95% CI for the predictive scores by using HLA binding algorithms and the TAP algorithm, demonstrated—for the first time—predictive score ranges, with statistical confidence. A recent study reported that prediction with these algorithms could not always be verified with experimental data(16,60,61). However, analytic methods and results on the 95% CIs for the experimentally identified epitopes have proven useful in the selection of predicted epitopes for further experimental verification.

The inventor found, for the first time, that: 1) the major difference between immunodominant tumor antigen epitopes and viral antigen epitopes lies in the structural features of proteolytic sites, but not in that for TAP binding and HLA binding; and 2) the proteasomes and related peptidases, at least the cleaving efficiencies of those enzymes, in the generation of HLA-A2.1-restricted tumor antigen epitopes may be different from those of viral antigen epitopes. future work may further expand this study to compare these two groups of epitopes presented by other MHC alleles when more documented epitopes are reported. Previous studies showed that viral antigen epitopes are preferentially processed by immunoproteasomes, while most tumor antigen epitopes are processed by constitutive proteasomes (62). Since results emphasize the critical role of proteasomes and related peptidases in the regulation of tumor antigen epitope processing and anti-tumor immune responses, the mechanisms underlying novel therapy of proteasome inhibition(63) in the modulation of tumor antigen epitope processing and anti-tumor immune responses remain an interesting topic in this field of research.

The results diverged from the previously published analysis of 274 epitope flanking regions (17), which did not identify differences in amino acid occurrence in epitope flanking regions. The discrepancy is likely due to the fact that the previous study only collected naturally processed peptides. The non-immunodominant epitopes that can be eluted from HLA may not join anti-tumor immune responses but may be functional in maintaining T cell repertoire (64,65). Therefore, the summarization of all diverted antigen peptides with potential immunodominance and that without immunodominance might average-out potential differences.

Of note, the noted differences could be caused by viral interference with antigen processing (66). However, recent reports on proteasomes and related proteases of antigen processing also support and explain findings (62). In viral infections, expression of IFN-gamma is induced by cytokines IL-2, IL-18, and IFN-alpha/beta, or by stimulation through TCR or natural killer (NK) cell receptors (6). Consequently, IFN-gamma alters proteasome activity quantitatively, by incorporating three immunosubunits, LMP2 (ibeta1), LMP7 (ibeta5), and MECL-1 (ibeta2), to replace the constitutive beta1 (delta), beta2 (MB1), and beta5 (Z) subunits in 20S core proteasome. Thus, two types of proteasome exist, “constitutive proteasomes,” which are present in all somatic cells, and “immunoproteasomes,” which are expressed under the influence of cytokines such as IFN-gamma (7). In addition, IFN-gamma also upregulates the expression of two other proteins, PA28alpha and PA28beta, which form the heptameric proteasome activator complex PA28 (67). In contrast with virally infected cells, in a large number of tumors the expression of IFN-gamma-induced proteasome subunits LMP2 and LMP7 is downregulated (68), suggesting that processing of tumor antigen epitopes may be different from those of viral antigen epitopes (7). In conjunction with these findings, results, via analyzing the substrates for proteolytic enzymes with unique bioinformatic approach, indicate that half of C-terminal cleavage sites in the tumor antigen epitopes are not shared by the viral epitopes, suggesting the possibility that more proteasomes than immunoproteasomes mediate cleavage of the C-terminus of tumor antigen epitopes; whereas, more immunoproteasomes are involved in production of the C-terminus of viral antigen epitopes. In addition to proteasomes, the parallel system potentially contributing to differences in the generation of tumor antigen and viral antigen epitopes is tripeptidyl peptidase II (TRPII; EC3.4.14.10), which is able to generate the HLA-restricted HIV Nef epitope independently of proteasomes (69). This parallel system may also contribute to differences between the two groups in the C-terminal cleavage sites.

The results indicate that, in comparison with the C-terminal cleavage sites of the epitopes, the differences in N-terminal cleavage sites between the two groups of epitopes were even larger. In addition, the inventor found that the most frequently occurring amino acid pairs in both N-terminal (Pn1-Pn1′) and C-terminal cleavage sites (Pc1-Pc1′) in the generation of tumor antigen epitopes are different from those of the viral antigen epitopes. It has been reported that many antigenic peptides are generated as amino-terminal extended precursor peptides, and that these require amino-terminal trimming by aminopeptidases located either in the cytosol or in the endoplasmic reticulum (ER)(8, 70-72). It should be noted that the expression of both cytosolic leucine aminopeptidase (EC3.4.11.1)(8) and ER aminopeptidase I (ERAP1)(73) is IFN-gamma induced, suggesting that such expression may be involved more in the generation of viral antigen rather than tumor antigen epitopes, which is similar to the function of immunoproteasomes (7). In conjunction with these reports, results showed that only one fifth of the N-terminal cleavage sites of the tumor antigen epitopes overlap with those of the viral antigen epitopes. The differences between the two groups of epitopes may result from different enzymatic activities; it is more likely that the N-terminal cleavage sites of viral epitopes would be generated by IFN-gamma-induced aminopeptidases; whereas, the N-terminus of the tumor antigen epitopes is most likely cleaved by IFN-gamma insensitive aminopeptidases. It is well documented that IFN-gamma plays a critical role in mounting anti-tumor immune responses (74). Along with this finding, bioinformatic results with statistical significance suggest that future tumor antigen-specific immunotherapy complemented by IFN-gamma may enhance the processing and presentation of MHC class I-restricted tumor antigen epitopes via upregulation of immunoproteasomes and IFN-gamma-induced peptidases.

### HLA Restricted T Cell Reactive Epitope Nucleic Acids

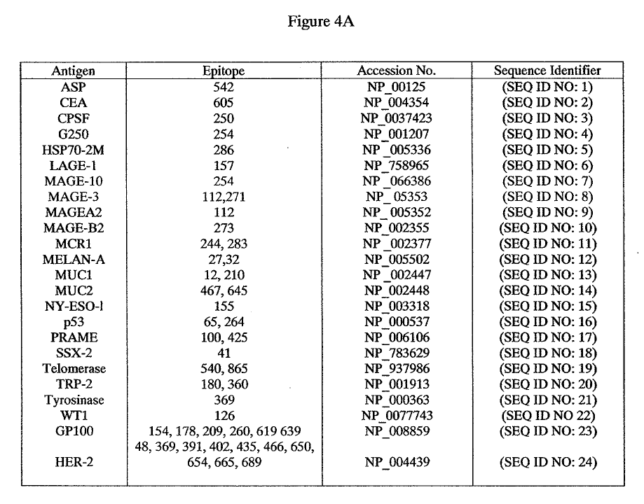

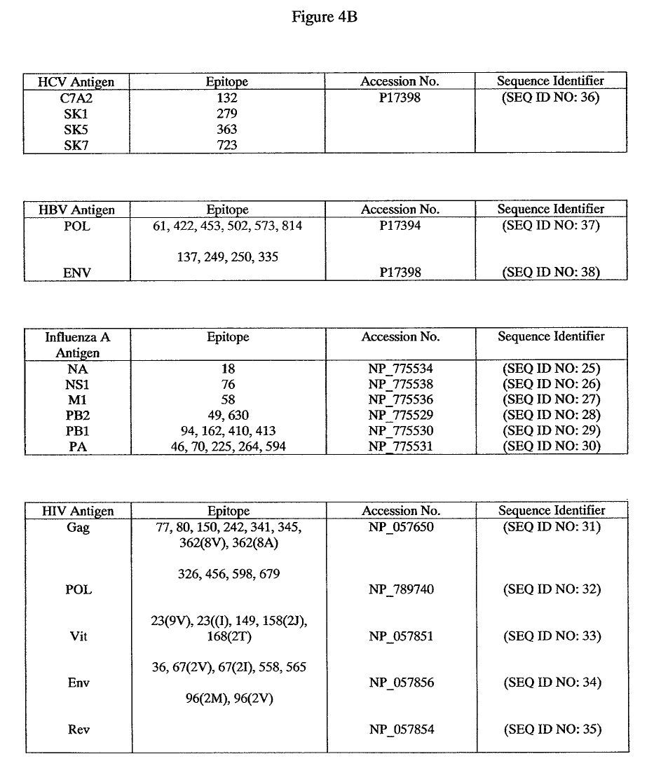

One aspect of the present invention are the polynucleotide sequences encoding, for example, the polypeptide sequences essentially as set forth in SEQ ID NOs: 1-38, the complement of these sequences, the RNA versions of both DNA strands and the information otherwise contained within the linear sequence of these polynucleotide sequences, and fragments thereof. In the case of nucleic acid segments, sequences for use with the present invention are those that have greater than about 50 to 60% homology with any portion of the polynucleotide sequences described herein, sequences that have between about 61% and about 70%; sequences that have between about 71 and about 80%; or between about 81% and about 90%; or between 91% and about 99%; or which contain nucleotides that are identical, functionality equivalent, or functionally irrelevant, with respect to the nucleotides encoding, for example, SEQ ID NOs: 1-38, or the antigens as set forth in FIG. 4, are considered to be essentially similar. Also encompassed within the present invention are nucleic acids that encode polypeptides that are at least 40% identical or similar to the amino acid sequences shown in SEQ ID NOs: 1-38, or FIG. 4.

The invention also encompasses other nucleic acids or nucleic acid like molecules that are sufficient in any regard to mimic, substitute for, or interfere with the HLA restricted T cell reactive epitope polypeptide sequences, as exemplified by SEQ ID NO: 1-38 or FIG. 4, or fragments thereof. It will also be understood that the nucleic acid and amino acid sequences may include additional residues, such as additional 5′- or 3′-sequences, and yet still be essentially as set forth in one of the sequences disclosed herein, so long as the sequence meets the criteria set forth, including the maintenance of functionality, or for the purpose of engineering altered functionality with respect to HLA restricted T cell reactive epitope.

Included within the invention are DNA or RNA segments including oligonucleotides, polynucleotides and fragments thereof, including DNA or RNA or nucleic acid-like sequences of genomic or synthetic origin, single or double stranded. The invention includes nucleic acid molecules, or nucleic acid-like molecules that are able to hybridize to the sequences encoding, for example, the epitopes of SEQ ID NOs: 1-38 or FIG. 4, under stringent or under permissive hybridization conditions, or to the complement of said sequences.

The invention also includes oligonucleotide, or oligonucleotide-like sequences such as phosphorthioate, or peptide nucleic acid sequences, which possess sufficient similarity with the sequences disclosed herein such that they are able to stably hybridize to the disclosed sequences, or their complements. Such sequences may be intended as antisense regulators of gene expression, or for the selective amplification or extension of adjoining sequences, for instance by PCR using a given annealing temperature, as would be determined by someone skilled in the art. In addition to the sequences disclosed here, related sequences in other organisms, or homologs, will be readily identified by hybridization using the present sequences. Similar techniques will also apply to the identification of mutant alleles, polymorphisms, deletions, insertions, and so forth, in genomic and cDNA sequences. Whole orpartial sequences referred to above may also be identified and isolated using techniques that involve annealing of short oligonucleotides to complementary sequences, such as those as might be present in the genomic DNA of a particular organism, or in genomic or cDNA, including expression cDNA, libraries. Thus, PCR is used to obtain DNA sequences homologous to, and which lie between, two primers, usually between 15 to 30 nucleotides which have annealing temperatures typically between 60-80 degrees Celsius may be substantially purified.

It will be understood that this invention is not limited to the particular nucleic acid sequences presented herein. Recombinant vectors, including for example plasmids, phage, viruses, and other sequences, and isolated DNA or RNA segments may therefore variously include the HLA restricted T cell reactive epitope gene sequences or their complements, and coding regions, as well as those that may bear selected alterations or modifications that nevertheless include HLA restricted T cell reactive epitope segments or may encode biologically or experimentally relevant amino acid sequences. Such sequences may be created by the application of recombinant DNA technology, where changes are engineered based on the consideration of the nucleotides or amino acids being exchanged, deleted, inserted, fused, or otherwise modified.

### HLA Restricted T Cell Reactive Epitope Proteins and Polypeptides

One aspect of the invention is the protein, polypeptide, oligopeptide, or amino acid sequences or fragments thereof, of HLA restricted T cell reactive epitopes, essentially as set forth in SEQ ID NOs: 1-38, or FIG. 4. The HLA restricted T cell reactive epitope polypeptides are exemplified by SEQ ID NOs: 1-38, or FIG. 4. Sequences that have greater than about 40-50% homology with any portion of the amino acid sequences described herein, sequences that have between about 51% and about 60%; sequences that have between about 61% and about 70% sequences that have between about 70 and about 80%; or between about 81% and about 90%; or between 91% and about 99%; or those that contain amino acids that are identical, functionally equivalent, or functionally irrelevant, for instance those specified by conservative, evolutionarily conserved, and degenerate substitutions, with respect to the amino acid sequences presented in, for example, SEQ ID NO: 1-38, or FIG. 4 are included. The invention thus applies to HLA restricted T cell reactive epitope polypeptide sequences, or fragments thereof, and nucleic acids which encode such polypeptides, such as those of other species. Reference is particularly, but not exclusively, made to the conserved regions of HLA restricted T cell reactive epitope, in contrast to similarity throughout the entire length. The invention thus encompasses amino acid sequences, or amino acid-like molecules, that are sufficient in any regard to mimic, substitute for, or interfere with the HLA restricted T cell reactive epitope amino acid sequences, or fragments thereof.

The invention encompasses HLA restricted T cell reactive epitope amino acid sequences that have been altered in any form, either through the use of recombinant engineering, or through post-translational or chemical modifications, including those that may be produced by natural, biological, artificial, or chemical methods. Naturally, it will be understood that this invention is not limited to the particular amino acid sequences presented herein. Altered amino acid sequences include those which have been created by the application of recombinant technology such that specific residues, regions, or domains have been altered, and which may be functionally identical, or which may possess unique biological or experimental properties with regards to function or interactions with natural and artificial ligands.

For instance such modifications may confer longer or shorter half-life, reduced or increased sensitivity to ligands that modify function, ability to detect or purify polypeptides, solubility, and so forth. Alternatively, such sequences may be shorter oligopeptides that possess an antigenic determinant, or property that interferes, or competes, with the function of a larger polypeptide, and those that affect interactions between HLA restricted T cell reactive epitope other proteins, other nucleic acid regions, and other proteins. Such sequences may be created by the application of the nucleotides or amino acids being exchanged, deleted, inserted, fused, or otherwise modified. Likewise, the current invention within, the sequences that may be naturally present as extensions of, or insertions within, the sequences disclosed herein, including alternative or longer N- and C-terminal sequences, or alternatively spliced protein isoforms.

Production and purification of polypeptides may be achieved in any of a variety of expression systems known to those skilled in the art, including recombinant DNA techniques, genetic recombination, and chemical synthesis. For instance, expression in prokaryotic cells may be achieved by placing protein coding nucleic sequences downstream of a promoter, such as T7, T3, lacI, lacZ, trp, or other cellular, viral, or artificially modified promoters including those that may be inducible by IPTG, tetracycline, maltose, and so forth. Such promoters are often provided for in commercially available recombinant DNA vectors such as pRSET ABC, pBluescript, pKK223-3, and others, or are easily constructed to achieve such a purpose, and often include the presence of multiple cloning sites (MCS) to facilitate typically contain efficient ribosome binding sites, and in some cases transcription termination signals.

Peptides, oligopeptides and polypeptides may also be produced by chemical synthesis, for instance solid phase techniques, either manually or under automated control such as Applied Biosystems 431 peptide synthesizer (Perkin Elmer). After synthesis, such molecules are often further purified by preparative high performance liquid chromatography. Thus, the invention provides methods for the production of epitopes for antibody production, or the production of small molecules that enhance or interfere with a specific function or interaction of the HLA restricted T cell reactive epitope polypeptides.

Methods to produce and purify said polypeptides in eukaryotic systems are widely available and understood by those proficient in the art. Cells for such production are known to include yeast and other fungi, Drosophila and Sf9 cells, cells of other higher eukaryotic organisms such as HeLa, COS, CHO and others, as well as plant cells. Similarly, expression could be achieved in prokaryotic or eukaryotic extracts that are able to translate RNAs into proteins, such as rabbit reticulocyte lysates.

### Vectors

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC®. 2.0 from INVITROGEN® and BACPACK®. BACULOVIRUS EXPRESSION SYSTEM FROM CLONTECH

Vectors may be of bacterial origin, which may comprise a promoter of a bacteriophage such as phage or T7 which is capable of functioning in the bacteria. In one of the most widely used expression systems, the nucleic acid encoding the HLA restricted T cell reactive epitope may be transcribed from the vector by T7 RNA polymerase (Studier et al, Methods in Enzymol. 185: 60-89, 1990). In the E. coli BL21 (DE3) host strain, used in conjunction with pET vectors, the T7 RNA polymerase is produced from the 1-lysogen DE3 in the host bacterium, and its expression is under the control of the IPTG inducible lac UV5 promoter. This system has been employed successfully for over-production of many proteins. Alternatively, the polymerase gene may be introduced on a lambda phage by infection with an int-phage such as the CE6 phage, which is commercially available (Novagen, Madison, USA). Other vectors include vectors containing the lambda PL promoter such as PLEX® (Invitrogen, NL), vectors containing the trc promoters such as pTrcH is Xpress® (Invitrogen), or pTrc99 (Pharmacia Biotech, SE), or vectors containing the tac promoter such as pKK223-3 (Pharmacia Biotech), or PMAL (New England Biolabs, MA, USA).

One of skill in the art will understand that cloning also requires the step of transforming a host cell with a recombinant nucleic acid molecule. A host cell is “transformed” by a nucleic acid when the nucleic acid is translocated into the cell from the extracellular environment. Any method of transferring a nucleic acid into the cell may be used; the term, unless otherwise indicated herein, does not imply any particular method of delivering a nucleic acid into a cell, nor that any particular cell type is the subject of transfer. For example, bacterial host cells, such as E. coli HB101, can be transformed by electroporation using any commercially-available electroporation apparatus known in the art, such as a GenePulser apparatus (Bio-Rad, Hercules, Calif.). In one embodiment, mammalian cells, such as BHK-21 cells or Vero cells (ATCC CCL-81), are transformed with a recombinant plasmid containing a cloned cDNA by the method of “transfection.” The term “transfection” refers to the transfer of genetic material into a eukaryotic cell, such as a mammalian cell, from the external environment of the cell.

One of skill in the art will appreciate the variety of methods of transfection that are available in the art. Such methods include the nucleic acid/CaPO4 co-precipitation method, the diethylaminoethyl (DEAE)-dextran method, the polybrene method, the cationic liposome method (“lipofection”), the electroporation method, the microinjection method, and the microparticle bombardment method. A description of transfection methods can be found in M. A. Aitken et al., Molecular Biomethods Handbook, Chapter 20, p. 235-250.

According to another embodiment of the instant invention, in vitro transcription is carried out on a recombinant plasmid carrying a cloned cDNA of the invention, under the control of an expressible promoter (i.e., a promoter which is effectively enabled or activated in vitro in the presence of corresponding transcription factors and RNA polymerase). The transcription process generates a fully-infectious mRNA transcript that can be used to transfect (i.e., infect) a cell host, such as BHK-21 (hamster kidney cells) or Vero cells. In one embodiment, the cDNA is operably linked with the bacteriophage transcriptional promoter, T7; to enable the in vitro transcription of the cDNA using bacteriophage T7 DNA-dependent RNA polymerase. One of ordinary skill in the art will appreciate that any suitable promoter, such as, for example, SP6, T3, any bacterial, viral, phage, or eukaryotic promoter, for controlling the transcription of, for example, the HLA restricted T cell reactive epitope gene, or fragment thereof, and for controlling the expression of a nucleotide sequence encoding a reporter is contemplated by the present invention. It will be appreciated that the promoter is typically selected from promoters which are functional in mammalian cells susceptible to infection by the HLA restricted T cell reactive epitope gene, or fragment thereof, encoding sequences of the invention, although prokaryotic orphage promoters and promoters functional in other eukaryotic cells may be used. The promoter is typically derived from promoter sequences of viral or eukaryotic genes. For example, it may be a promoter derived from the genome of a cell in which expression or transcription of, for example, the HLA restricted T cell reactive epitope gene, or fragment thereof, encoding sequence or construct is to occur.

With respect to eukaryotic promoters, they may be promoters that function in a ubiquitous manner (such as promoters of α-actin, β-actin, tubulin) or, alternatively, a tissue-specific manner (such as promoters of the genes for pyruvate kinase). Tissue-specific or cell-specific promoters specific for lymphocytes, dendritic cells, skin, brain cells and epithelial cells, for example the CD2, CD11c, keratin 14, Wnt-1 and Rhodopsin promoters, respectively. Preferably the epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter, the human cytomegalovirus (CMV) IE promoter, or SV40 promoter.

It may also be advantageous for the promoters to be inducible so that the levels of expression of, for example, the HLA restricted T cell reactive epitope gene, or fragment thereof encoding sequence can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above. It will be appreciated that the sources of promoter sequences, which typically can be retrieved using recombinant techniques from different cloning vectors and plasmids, etc., can be obtained from commercial sources, such as, NEW ENGLAND BIOLABS, INC. (MA), PROMEGA CORPORATION (WI), or BD BIOSCIENCES (CA), or from the laboratories of academic research groups upon request.

The invention also relates to cells which contain such recombinant constructs, where the host cell refers to mammalian, plant, yeast, insect, or other eukaryotic cells, or to prokaryotic, or archae, and vectors that are designed for a given host. Promoter-vector combinations could be chosen by a person skilled in these arts. In some cases, the desired outcome may not be protein, but RNA, and recombinant vectors would include those with inserts present in either forward or reverse orientations.

Many of the vectors and hosts have specific features that facilitate expression or subsequent purification. For instance DNA sequences to be expressed as proteins often appear as fusion with unrelated sequences that encode polyhistidine tags, or HA, FLAG, myc and other epitope tags for immunochemical purification and detection, or phosphorylation sites, or protease recognition sites, or additional protein domains such as glutathione S-transferase (GST), maltose binding protein (MBP), and so forth which facilitate purification. Vectors may also be designed which contain elements for polyadenylation, splicing and termination, such that incorporation of naturally occurring genomic DNA sequences that contain introns and exons can be produced and processed, or such that unrelated introns and other regulatory signals require RNA processing prior to production of mature, translatable RNAs. Proteins produced in the systems described above could be subject to a variety of post-translational modifications, such as glycosylation, phosphorylation, nonspecific or specific proteolysis or processing. Purification of HLA restricted T cell reactive epitope, or variants produced as described above can be achieved by any of several widely available methods. Cells may be subject to freeze-thaw cycles or sonication to achieve disruption, or may be fractionated into subcellular components prior to further purification. Purification may be achieved by one or more techniques such as precipitation with salts or organic solvents, ion exchange, hydrophobic interaction, HPLC and FPLC chromatograpic techniques. Affinity chromatographic techniques could include the use of polyclonal or monoclonal antibodies raised against the expressed polypeptide, or antibodies raised against or available for an epitopic tag such as HA or FLAG. Similarly, purification can be aided by affinity chromatography using fusions to the desired proteins such as GSH-affinity resin, maltose affinity resin, carbohydrate (lectin) affinity resin or, in a one embodiment, Ni-affinity resin, and so forth. In some instances purification is achieved in the presence of denaturing agents such as urea or guanidine, and subsequent dialysis techniques may be required to restore functionality, if desired.

Any method of in vitro transcription known to one of ordinary skill in the art is contemplated by the instant invention. It will be understood that the method of in vitro transcription of a DNA sequence relies on the operable linkage to an appropriate promoter and that the cognate RNA polymerase is used to direct transcription of the DNA starting at the promoter sequence. It will be further appreciated that the RNA polymerase and promoter can be of bacterial, eukaryotic, or viral (including bacteriophage) origin. Bacteriophage-RNA polymerases are very robust, and the availability of purified recombinant proteins facilitates the generation of large quantities of RNA from cloned cDNA sequences. In contrast, eukaryotic in vitro transcription systems yield relatively small quantities of RNA. Bacteriophage-RNA polymerases, such as from bacteriophages SP6, T7, and T3, are especially suitable for the generation of RNA from DNA sequences cloned downstream of their specific promoters because, first, their promoters are small and easily incorporated into plasmid vectors and second, the polymerases are quite specific for their cognate promoters, which results in very little incorrect transcriptional initiation from DNA templates. Any suitable promoter, however, is contemplated by the instant invention, including, for example, bacterial, phage, viral, and eukaryotic promoters. Strong termination sequences are not available for these polymerases so that DNA templates can be linearized with a restriction enzyme 3′ to the desired end of the RNA transcript and the polymerase is forced to stop at this point-a process referred to as “run-off” transcription. A full description of in vitro transcription can be found in M. A. Aitken et al., Molecular Biomethods Handbook, Chapter 26, p. 327-334 and Sambrook, J. and D. W. Russell, Molecular Cloning: A Laboratory Manual, Third Edition (2001).

U.S. Pat. No. 6,143,502 (Grentzmann et al.) A dual luciferase reporter system for measuring recoding efficiencies in vivo or in vitro from a single construct has been designed (FIG. 3). The firefly luciferase gene (fluc) has been cloned behind the renilla luciferase gene (rluc) into an altered vector pRL-SV40 vector (Promega Corp., Madison, W is; catalog no. TB239). Expression features for initiation and termination of transcription and translation, as well as the nature of the two reporter genes (short enough to be efficiently synthesized in an in vitro translation system), allow application of the same reporter construct for in vivo and in vitro applications. Between the 5′ reporter (rluc) and the 3′ reporter (fluc) two alternative polylinkers have been inserted, yielding p2luc and p2luci. The p2luc polylinker has restriction sites for digestion with SalI, BamHI, and SacI, whereas the p2luci polylinker has restriction sites for digestion with SalI, ApaI, BglII, Eco47III, BamHI, SmaI, and SacI. The assay using these reporter plasmids combines rapidity of the reactions with very low background levels and provides a powerful assay. In vitro experiments can be performed in 96-well microtiter plates, and in vivo experiments can be performed in 6-well culture dishes. This makes the dual-luciferase assay suitable for high throughput screening approaches.

### Host Cells

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE®. Competent Cells and SOLOPACK® Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12, etc. Many host cells from various cell types and organisms are available and would be known to one of skill in the art. Similarly, a viral vector may be used in conjunction with either a eukaryotic or prokaryotic host cell, particularly one that is permissive for replication or expression of the vector.

### Systems and Kits

A diagnostic system in kit form of the present invention includes, in an amount sufficient for at least one assay, a polypeptide, antibody composition or monoclonal antibody composition of the present invention, as a packaged reagent. Instructions for use of the packaged reagent are also typically included.

The invention provides a delivery system in kit form which includes, in an amount sufficient for at least one administration, of a nucleic acid, a polypeptide, antibody composition, or monoclonal antibody composition of the present invention in packaged form. Instructions for use of the packaged composition are also typically included.

As used herein, the term “package” refers to a solid matrix or material such as glass, plastic, paper, foil and the like capable of holding within fixed limits a polypeptide, antibody composition or monoclonal antibody composition of the present invention. Thus, for example, a package can be a glass vial used to contain milligram quantities of a contemplated polypeptide or it can be a microtiter plate well to which microgram quantities of a contemplated polypeptide have been operatively affixed, i.e., linked so as to be capable of being immunologically bound by an antibody. “Instructions for use” typically include a tangible expression describing the reagent concentration or at least one assay method parameter such as the relative amounts of reagent and sample to be admixed, maintenance time periods for reagent/sample admixtures, temperature, buffer conditions and the like.

In preferred embodiments, a diagnostic system of the present invention further includes a label or indicating means capable of signaling the formation of a complex containing a polypeptide or antibody molecule of the present invention. The word “complex” as used herein refers to the product of a specific binding reaction such as an antibody-antigen or receptor-ligand reaction. Exemplary complexes are immunoreaction products.

As used herein, the terms “label” and “indicating means” in their various grammatical forms refer to single atoms and molecules that are either directly or indirectly involved in the production of a detectable signal to indicate the presence of a complex. Any label or indicating means can be linked to or incorporated in an expressed protein, polypeptide, or antibody molecule that is part of an antibody or monoclonal antibody composition of the present invention, or used separately, and those atoms or molecules can be used alone or in conjunction with additional reagents such labels are themselves well-known in clinical diagnostic chemistry and constitute a part of this invention only insofar as they are utilized with otherwise novel proteins methods and/or systems. The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyante (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC) and the like. A description of immunofluorescence analysis techniques is found in DeLuca, “Immunofluorescence Analysis”, in Antibody As a Tool, Marchalonis, et al., eds., John Wiley & Sons, Ltd., pp. 189-231 (1982), which is incorporated herein by reference.

In preferred embodiments, the indicating group is an enzyme, such as horseradish peroxidase (HRP), glucose oxidase, or the like. In such cases where the principal indicating group is an enzyme such as HRP or glucose oxidase, additional reagents are required to visualize the fact that a receptor-ligand complex (immunoreactant) has formed. Such additional reagents for HRP include hydrogen peroxide and an oxidation dye precursor such as diaminobenzidine. An additional reagent useful with glucose oxidase is 2,2′-azino-di-(3-ethyl-benzthiazoline-G-sulfonic acid) (ABTS).

Radioactive elements are also useful labeling agents and are used illustratively herein. An examplary radiolabeling agent is a radioactive element that produces gamma ray emissions. Elements which themselves emit gamma rays, such as ¹²⁴I, ¹²⁵I, ¹²⁸I, ¹³²I and ⁵¹Cr represent one class of gamma ray emission-producing radioactive element indicating groups. Particularly preferred is ¹²⁵I. Another group of useful labeling means are those elements such as ¹¹C, ¹⁸F, ¹⁵O and ¹³N which themselves emit positrons. The positrons so emitted produce gamma rays upon encounters with electrons present in the animal's body. Also useful is a beta emitter, such ¹¹¹indium or ³H.

The linking of labels, i.e., labeling of, polypeptides and proteins is well known in the art. For instance, antibody molecules produced by a hybridoma can be labeled by metabolic incorporation of radioisotope-containing amino acids provided as a component in the culture medium. See, for example, Galfre et al., Meth. Enzymol., 73:3-46 (1981). The techniques of protein conjugation or coupling through activated functional groups are particularly applicable. See, for example, Aurameas, et al., Scand. J. Immunol., Vol. 8 Suppl. 7:7-23 (1978), Rodwell et al., Biotech., 3:889-894 (1984), and U.S. Pat. No. 4,493,795, which are all incorporated herein by reference.

The diagnostic kits of the present invention can be used in an “ELISA” format to detect, for example, the presence or quantity of HLA restricted T cell reactive epitope in a body fluid sample such as serum, plasma, or urine, etc. “ELISA” refers to an enzyme-linked immunosorbent assay that employs an antibody or antigen bound to a solid phase and an enzyme-antigen or enzyme-antibody conjugate to detect and quantify the amount of an antigen or antibody present in a sample. A description of the ELISA technique is found in Chapter 22 of the 4th Edition of Basic and Clinical Immunology by D. P. Sites et al., published by Lange Medical Publications of Los Altos, Calif. in 1982 and in U.S. Pat. No. 3,654,090; No. 3,850,752; and No. 4,016,043, which are all incorporated herein by reference.

Thus, in preferred embodiments, a polypeptide, antibody molecule composition or monoclonal antibody molecule composition of the present invention can be affixed to a solid matrix to form a solid support that comprises a package in the subject diagnostic systems.

The reagent is typically affixed to the solid matrix by adsorption from an aqueous medium although other modes of affixation, well known to those skilled in the art, can be used.

Useful solid matrices are also well known in the art. Such materials are water insoluble and include cross-linked dextran; agarose; beads of polystyrene beads about 1 micron to about 5 millimeters in diameter; polyvinyl chloride, polystyrene, cross-linked polyacrylamide, nitrocellulose- or nylon-based webs such as sheets, strips or paddles; or tubes, plates or the wells of a microtiter plate such as those made from polystyrene or polyvinylchloride.

The packaging materials discussed herein in relation to diagnostic systems are those customarily utilized in diagnostic systems. Such materials include glass and plastic (e.g., polyethylene, polypropylene and polycarbonate) bottles, vials, plastic and plastic-foil laminated envelopes and the like. In one embodiment a diagnostic system of the present invention is useful for assaying for the presence of HLA restricted T cell reactive epitope. Such a system comprises, in kit form, a package containing an antibody to HLA restricted T cell reactive epitope.

### Vaccine

Another aspect of the invention relates to a method for inducing an immunological response in an individual, particularly a mammal which comprises inoculating the individual with, for example, an HLA restricted T cell reactive epitope, or a fragment, or variant thereof, or combinations thereof. Also provided are methods whereby such immunological response slows bacterial replication. Yet another aspect of the invention relates to a method of inducing immunological response in an individual which comprises delivering to such individual a nucleic acid vector to direct expression of, for example, an HLA restricted T cell reactive epitope, or a fragment or a variant thereof, for expressing, for example, HLA restricted T cell reactive epitope, or a fragment or a variant thereof in vivo in order to induce an immunological response, such as, to produce antibody and/or T cell immune response, including, for example, cytokine-producing T cells or cytotoxic T cells, to protect said individual from disease, whether that disease is already established within the individual or not. One way of administering the gene is by accelerating it into the desired cells as a coating on particles or otherwise. Such nucleic acid vector may comprise DNA, RNA, a modified nucleic acid, or a DNA/RNA hybrid.

A further aspect of the invention relates to an immunological composition which, when introduced into an individual capable or having induced within it an immunological response, induces an immunological response in such individual to, for example, an HLA restricted T cell reactive epitope gene, or protein coded therefrom, wherein the composition comprises, for example, a recombinant HLA restricted T cell reactive epitope gene, or protein coded therefrom comprising DNA which codes for and expresses an antigen of said HLA restricted T cell reactive epitope or protein coded therefrom. The immunological response may be used therapeutically or prophylactically and may take the form of antibody immunity or cellular immunity such as that arising from CTL or CD4+T cells.

In an exemplary embodiment, an HLA restricted T cell reactive epitope polypeptide or a fragment thereof may be fused with co-protein which may not by itself produce antibodies, but is capable of stabilizing the first protein and producing a fused protein which will have immunogenic and protective properties. Thus fused recombinant protein, preferably further comprises an antigenic co-protein, such as lipoprotein D from Hemophilus influenzae, Glutathione-S-transferase (GST) or beta-galactosidase, relatively large co-proteins which solubilize the protein and facilitate production and purification thereof. Moreover, the co-protein may act as an adjuvant in the sense of providing a generalized stimulation of the immune system. The co-protein may be attached to either the amino or carboxy terminus of the first protein.

Provided by this invention are compositions, particularly vaccine compositions, and methods comprising the polypeptides or polynucleotides of the invention and immunostimulatory DNA sequences, such as those described in Sato, Y. et al. Science 273: 352 (1996).

The invention also includes a vaccine formulation which comprises an immunogenic recombinant protein of the invention together with a suitable carrier. Since the protein may be broken down in the stomach, it is preferably administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteriostats and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

While the invention has been described with reference to certain HLA restricted T cell reactive epitope protein, it is to be understood that this covers fragments of the naturally occurring protein and similar proteins with additions, deletions or substitutions which do not substantially affect the immunogenic properties of the recombinant protein.

### Target Antigens

An embodiment of the present invention relates to an antibody that binds to a HLA restricted T cell reactive epitope protein. Typical amino acid sequences of HLA restricted T cell reactive epitope protein are exemplified in SEQ ID NOs: 1-38, or FIG. 4. That is, an antibody according to the first embodiment of the present invention is preferably an antibody that specifically binds to, for example, the HLA restricted T cell reactive epitope polypeptide. Full length HLA restricted T cell reactive epitope protein are exemplified in SEQ ID NOs: 1-38, or FIG. 4, and variants, fragments, muteins, etc., and those proteins derived from this protein. It is known that humans have a diversity of allele mutations and those proteins with one or more amino acids substituted, deleted, inserted, or added are also included in the HLA restricted T cell reactive epitope protein. However, it is not limited to these. It is known that humans have a diversity of allele mutations and those proteins with one or more amino acids substituted, deleted, inserted, or added are also included in the HLA restricted T cell reactive epitope protein. However, it is not limited to these.

Fragments of the HLA restricted T cell reactive epitope protein may serve as the target antigen for the antibody binding. These antigen fragments may be about 6, 7, 8, 9, 10, 11, 12, 13, 14, 15, 16, 17, 18, 19, or 20 amino acids in length. The antigen fragments may by about 10, 20, 30, 40, 50, or 100 amino acids in length. The antibody of the present invention may be either a polyclonal antibody or a monoclonal antibody. To specifically detect a high molecular weight soluble HLA restricted T cell reactive epitope protein, it is desirable to use antibodies to certain limited epitopes and hence monoclonal antibodies are preferable. Molecule species are not particularly limited. Immunoglobulins of any class, subclass or isotype may be used.

### Antibodies and Antibody Compositions

Additionally, the present invention includes a purified antibody produced in response to immunization with HLA restricted T cell reactive epitopes, as well as compositions comprising this purified antibody.

Antibodies refer to single chain, two-chain, and multi-chain proteins and glycoproteins belonging to the classes of polyclonal, monoclonal, chimeric, and hetero immunoglobulins; it also includes synthetic and genetically engineered variants of these immunoglobulins. “Antibody fragment” includes Fab, Fab′, F(ab′)2, and Fv fragments, as well as any portion of an antibody having specificity toward a desired target epitope or epitopes. A humanized antibody is an antibody derived from a non-human antibody, typically murine, that retains or substantially retains the antigen-binding properties of the parent antibody but which is less immunogenic in humans, U.S. Pat. No. 5,530,101, incorporated herein by reference in its entirety.

An antibody composition of the present invention is typically produced by immunizing a laboratory mammal with an inoculum of the present invention and to thereby induce in the mammal antibody molecules having the appropriate polypeptide immunospecificity. The polyclonal antibody molecules are then collected from the mammal and isolated to the extent desired by well known techniques such as, for example, by immunoaffinity chromatography. The antibody composition so produced can be used in, inter alia, the diagnostic methods and systems of the present invention to detect HLA restricted T cell reactive epitope in a body sample.

Monoclonal antibody compositions are also contemplated by the present invention. A monoclonal antibody composition contains, within detectable limits, only one species of antibody combining site capable of effectively binding HLA restricted T cell reactive epitope. Thus, a monoclonal antibody composition of the present invention typically displays a single binding affinity for HLA restricted T cell reactive epitope even though it may contain antibodies capable of binding proteins other than HLA restricted T cell reactive epitope. Suitable antibodies in monoclonal form, typically whole antibodies, can also be prepared using hybridoma technology described by Niman et al., Proc. Natl. Sci., U.S.A., 80:4949-4953 (1983), which description is incorporated herein by reference. Briefly, to form the hybridoma from which the monoclonal antibody composition is produced, a myeloma or other self-perpetuating cell line is fused with lymphocytes obtained from the spleen of a mammal hyperimmunized with a polypeptide of this invention.

The antibody compositions produced by the above method can be used, for example, in diagnostic and therapeutic modalities wherein formation of an HLA restricted T cell reactive epitope-containing immunoreaction product is desired.

### Diagnostic Use

In another embodiment of the present invention, measurement of HLA restricted T cell reactive epitope, or proteins which are immunologically related to HLA restricted T cell reactive epitope, can be used to detect and/or stage a disease or disorder in a subject. The measured amount of the soluble molecule or of the total marker is compared to a baseline level. This baseline level can be the amount which is established to be normally present in the body fluid of subjects with various degrees of the disease or disorder. An amount present in the body fluid of the subject which is similar to a standard amount, established to be normally present in the body fluid of the subject during a specific stage of the disease or disorder, is indicative of the stage of the disease in the subject. The baseline level could also be the level present in the subject prior to the onset of disease or the amount present during remission of disease, or from individuals not afflicted with the disease or condition.

The present invention also provides for the detection or diagnosis of disease or the monitoring of treatment by measuring the amounts of HLA restricted T cell reactive epitope transcript or peptide in a sample before and after treatment, and comparing the two measurements. The change in the levels of the markers relative to one another can be an improved prognostic indicator. A comparison of the amounts of a total marker with the amount of intra-cytoplasmic marker or membrane-bound marker is also envisioned.

The present invention provides a method for monitoring the effect of a therapeutic treatment on a subject who has undergone the therapeutic treatment. This method comprises measuring at suitable time intervals the amount of a soluble molecule or soluble fragment thereof, or the amount of HLA restricted T cell reactive epitopes or fragment thereof. Any change or absence of change in the amount of the soluble molecule or in the amount of the HLA restricted T cell reactive epitope can be identified and correlated with the effect of the treatment on the subject. In a specific embodiment of the invention, soluble molecules immunologically related to HLA restricted T cell reactive epitopes can be measured in the serum of patients by a sandwich enzyme immunoassay (for an example) in order to predict disease prognosis, for example, in viral infections, inflammation, autoimmune diseases, and tumors, or to monitor the effectiveness of treatments such as anti-viral administration.

### Pharmaceutical Compositions and Administration

Administration of therapeutically effective amounts is by any of the routes normally used for introducing protein or encoding nucleic acids into ultimate contact with the tissue to be treated. The protein or encoding nucleic acids are administered in any suitable manner, preferably with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remington's Pharmaceutical Sciences, 17th ed. 1985)).

The protein or encoding nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

In certain cases, alteration of a genomic sequence in a pluripotent cell (e.g., a hematopoietic stem cell) is desired. Methods for mobilization, enrichment and culture of hematopoietic stem cells are known in the art. See for example, U.S. Pat. Nos. 5,061,620; 5,681,559; 6,335,195; 6,645,489 and 6,667,064.

The invention will be illustrated in more detail with reference to the following Examples, but it should be understood that the present invention is not deemed to be limited thereto.

### EXAMPLES

### Example 1

Experimentally identified HLA-A2.1-restricted tumor antigen epitopes and viral antigen epitopes. The 47 HLA-A2.1-restricted tumor antigen epitopes previously identified by the experimental approaches of others (18,19) are listed in Table I (also, see the web database at: www.cancerimmunity.org/peptidedatabase/Tcellepitopes.htm). The experimentally confirmed HLA-A2.1-restricted viral antigen epitopes are as follows: the 24 immunodeficiency virus (HIV) viral antigen epitopes, including those encoded by five HIV viral proteins collected from the HIV Molecular Immunology Database (hiv-web.lanl.gov/content/immunology/maps/ctl/p17.html), as well as published data(36); the 10 hepatitis B virus (HBV) epitopes encoded by two proteins of HBV (37); the four hepatitis C virus (HCV) epitopes encoded by four proteins (38); and the 14 influenza A virus epitopes encoded by six proteins(39). To achieve parity, only nonapeptide tumor antigen epitopes and nonapeptide viral antigen epitopes—and not epitopes of other lengths—were analyzed.

### Example 2

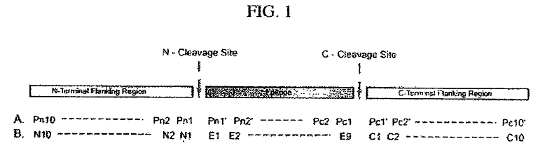

Nomination of amino acid positions in the antigen epitopes and flanking regions. In accordance with the enzymatic cleavage nomenclature of Schechter and Berger (40), analyses (FIG. 1) included the ten amino acid residues flanking the N-terminal cleavage site of the nonapeptide epitopes and the ten residues flanking the C-terminal cleavage site of the antigen epitopes(41). The protein-protein BLAST search for short exact matches was performed on the NCBI website (www.ncbi.nlm.nih.gov/BLAST/) to retrieve both the N-terminal and C-terminal flanking regions of HLA-A2.1-restricted T cell antigen epitopes. During the final phase, epitopes are generated by two cleavages on both the N-terminus and C-terminus; thus, in addition to enzymatic cleavage nomenclature(40), the amino acid positions in the epitope (E1 to E9), the N-terminal (N10 to N1), and C-terminal (C1 to C10C) flanking regions were further nominated (FIG. 1). Substrate specificities of the enzymes were retrieved from the Comprehensive Enzyme Information System, BRENDA (www.brenda.unikoeln.de/index.php4).

### Example 3

Algorithms for antigenic epitope prediction. The four algorithms, the MAPPP (www.mpiib-berlin.mpg.de/MAPPP/cleavage.html)(42), the MHC-Pathway (www.mhc-pathway.net/)(43), the MHC-Pathway immunoproteasome, and the NetChop3.0 neural network predictor (www.cbs.dtu.dk/services/NetChop/) (44), were used to predict the proteasome cleavage sites on antigens. In addition, a prediction algorithm (TAPPred) (www.imtech.res.in/raghava/tappred/) for the transporter associated protein (TAP) binding was used to predict the TAP binding potential of antigen epitopes. Furthermore, binding of the antigen peptide epitopes on the HLA-A2.1 molecule was predicted by using two different web-based algorithms, including one on the BIMAS/NIH (BIMAS) website (bimas.dcrt.nih.gov/molbio/hla_bind/) and another on the SYFPEITHI (SYF) website (syfpeithi.bmi-heidelberg.com/scripts/MHCServer.dll/home.htm).

### Example 4

Probability for the overlaps of amino acid pairs at the N- and C-terminal cleavage sites. The 400 potential pairs of amino acids in nature were numbered from 1 to 400. It was assumed that the amino acid pairs were represented in the cleavage sites of tumor antigens and virus antigens, which were assigned as the pair set, and the pair set, respectively, allowing for repetitions. The probability was calculated that there is an overlap between the sets such that the pairs from the first set are the same as the pairs in the second set (see FIG. 2A). Such an overlap as an ordered pair was denoted. A simple counting method, as described (45), was used to compute event probabilities (the probability mass function) for different overlaps for and.

### Example 5

Statistical Methods. Using a one-sample test for binomial proportion(46), the frequencies of amino acid residues in the N-terminal and C-terminal positions of the HLA-A2.1-restricted tumor antigen epitope cleavage sites were calculated and compared to the general occurrence frequencies of each amino acid in any position of the proteins(47). Similarly, the frequencies of amino acid residues in the N-terminal and C-terminal positions of both cleavage sites of the HLA-A2.1-restricted viral antigen epitopes were calculated and compared to the general occurrence frequencies of each amino acid in any position of the proteins(47). The Wilcoxon rank-sum test was used to compare proteasome cleavage probabilities between the tumor epitopes and the viral epitopes, as predicted with the MAPPP algorithm (www.mpiib-berlin.mpg.de/MAPPP/cleavage.html).

### REFERENCES

1. Finn O. J. 2003. Cancer vaccines: Between the idea and the reality. Nat. Rev. Immunol. 3:630.
2. Yang F., X. F. Yang. 2005. New concepts in tumor antigens: Their significance in future immunotherapies for tumors. Cell Mol. Immunol. 2:331.
3. Lollini P. L., F. Cavallo, P. Nanni, G. Formi. 2006. Vaccines for tumour prevention. Nat. Rev. Cancer 6:204.
4. De Visser K. E., T. N. Schumacher, A. M. Kruisbeek. 2003. Cd8+t cell tolerance and cancer immunotherapy. J. Immunother. 26:1.
5. Van Der Bruggen P., B. J. Van Den Eynde. 2006. Processing and presentation of tumor antigens and vaccination strategies. Curr. Opin. Immunol. 18:98.
6. Malmgaard L. 2004. Induction and regulation of ifns during viral infections. J. Interferon Cytokine Res. 24:439.
7. Kloetzel P. M., F. Ossendorp. 2004. Proteasome and peptidase function in mhcclass-i-mediated antigen presentation. Curr. Opin. Immunol. 16:76.
8. Bening a J., K. L. Rock, A. L. Goldberg. 1998. Interferon-gamma can stimulate post-proteasomal trimming of the n terminus of an antigenic peptide by inducing leucine aminopeptidase. J. Biol. Chem. 273:18734.
9. Rock K. L., I. A. York, A. L. Goldberg. 2004. Post-proteasomal antigen processing for major histocompatibility complex class i presentation. Nat. Immunol. 5:670.
10. Kloetzel P. M. 2004. Generation of major histocompatibility complex class i antigens: Functional interplay between proteasomes and tppii. Nat. Immunol. 5:661.
11. Geier E., G. Pfeifer, M. Wilm, M. Lucchiari-Hartz, W. Baumeister, K. Eichmann, G. Niedermann. 1999. A giant protease with potential to substitute for some functions of the proteasome. Science 283:978.
12. Luckey C. J., G. M. King, J. A. Marto, S. Venketeswaran, B. F. Maier, V. L. Crotzer, T. A. Colella, J. Shabanowitz, D. F. Hunt, V. H. Engelhard. 1998. Proteasomes can either generate or destroy mhc class i epitopes: Evidence for nonproteasomal epitope generation in the cytosol. J. Immunol. 161:112.
13. Uebel S., W. Kraas, S. Kienle, K. H. Wiesmuller, G. Jung, R. Tampe. 1997. Recognition principle of the tap transporter disclosed by combinatorial peptide libraries. Proc. Natl. Acad. Sci. U.S.A. 94:8976.
14. Daniel S., V. Brusic, S. Caillat-Zucman, N. Petrovsky, L. Harrison, D. Riganelli, F. Sinigaglia, F. Gallazzi, J. Hammer, P. M. Van Endert. 1998. Relationship between peptide selectivities of human transporters associated with antigen processing and hla class i molecules. J. Immunol. 161:617.
15. Bhasin M., G. P. Raghava. 2004. Analysis and prediction of affinity of tap binding peptides using cascade svm. Protein Sci. 13:596.
16. Pelte C., G. Cherepnev, Y. Wang, C. Schoenemann, H. D. Volk, F. Kern. 2004. Random screening of proteins for hla-a*0201-binding nine-amino acid peptides is not sufficient for identifying cd8 t cell epitopes recognized in the context of hla-a*0201. J. Immunol. 172:6783.
17. Altuvia Y., H. Margalit. 2000. Sequence signals for generation of antigenic peptides by the proteasome: Implications for proteasomal cleavage mechanism. J. Mol. Biol. 295:879.
18. Renkvist N., C. Castelli, P. F. Robbins, G. Parmiani. 2001. A listing of human tumor antigens recognized by t cells. Cancer Immunol. Immunother. 50:3.
19. Novellino L., C. Castelli, G. Parmiani. 2004. A listing of human tumor antigens recognized by t cells: March 2004 update. Cancer Immunol. Immunother.
20. Sahin U., O. Tureci, H. Schmitt, B. Cochlovius, T. Johannes, R. Schmits, F. Stenner, G. Luo, I. Schobert, M. Pfreundschuh. 1995. Human neoplasms elicit multiple specific immune responses in the autologous host. Proc. Natl. Acad. Sci. U.S.A. 92:11810.
21. Chen Y. T. 2000. Cancer vaccine: Identification of human tumor antigens by serex [in process citation]. Cancer J. Sci. Am. 6 Suppl. 3:S208.
22. Scanlan M. J., D. Jager. 2001. Challenges to the development of antigenspecific breast cancer vaccines. Breast Cancer Res. 3:95.
23. Rosenberg S. A. 2004. Shedding light on immunotherapy for cancer. N. Engl. J. Med. 350:1461.
24. Chen Y. 2004. Serex review. Cancer Immunity www.cancerimmunity.org/SEREX/
25. Wu C. J., X. F. Yang, S. Mclaughlin, D. Neuberg, C. Canning, B. Stein, E. P. Alyea, R. J. Soiffer, G. Dranoff, J. Ritz. 2000. Detection of a potent humoral response associated with immune-induced remission of chronic myelogenous leukemia. J. Clin. Invest. 106:705.
26. Yang X. F., C. J. Wu, S. Mclaughlin, A. Chillemi, K. S. Wang, C. Canning, E. P. Alyea, P. Kantoff, R. J. Soiffer, G. Dranoff, J. Ritz. 2001. Cml66, a broadly immunogenic tumor antigen, elicits a humoral immune response associated with remission of chronic myelogenous leukemia. Proc. Natl. Acad. Sci. U.S.A. 98:7492.
27. Yan Y., L. Phan, F. Yang, M. Talpaz, Y. Yang, Z. Xiong, B. Ng, N. A. Timchenko, C. J. Wu, J. Ritz, H. Wang, X. F. Yang. 2004. A novel mechanism of alternative promoter and splicing regulates the epitope generation of tumor antigen cml66-1. J. Immunol. 172:651.
28. Yang X. F., C. J. Wu, L. Chen, E. P. Alyea, C. Canning, P. Kantoff, R. J. Soiffer, G. Dranoff, J. Ritz. 2002. Cml28 is a broadly immunogenic antigen, which is overexpressed in tumor cells. Cancer Res. 62:5517.
29. Yan Y., Xiong, Z, Yang, F, Yang, Y, Ng, B, Lachman, L, Wang, H, Yang, X F. 2004. Broadly immunogenic serex antigen cml66-1 elicits humoral and cellular immune responses. FASEB J. 18(4): A58.
30. Rosenberg S. A. 2000. Identification of cancer antigens: Impact on development of cancer immunotherapies. Cancer J. Sci. Am. 6 Suppl. 3:S200.
31. Rosenberg S. A. 2001. Progress in human tumour immunology and immunotherapy. Nature 411:380.
32. Manici S., T. Sturniolo, M. A. Imro, J. Hammer, F. Sinigaglia, C. Noppen, G. Spagnoli, B. Mazzi, M. Bellone, P. Dellabona, M. P. Protti. 1999. Melanoma cells present a mage-3 epitope to cd4(+) cytotoxic t cells in association with histocompatibility leukocyte antigen dr11. J. Exp. Med. 189:871.
33. Van Der Bruggen P., Y. Zhang, P. Chaux, V. Stroobant, C. Panichelli, E. S. Schultz, J. Chapiro, B. J. Van Den Eynde, F. Brasseur, T. Boon. 2002. Tumor-specific shared antigenic peptides recognized by human t cells. Immunol. Rev. 188:51.
34. Nakatsura T., S. Senju, M. Ito, Y. Nishimura, K. Itoh. 2002. Cellular and humoral immune responses to a human pancreatic cancer antigen, coactosin-like protein, originally defined by the serex method. Eur. J. Immunol. 32:826.
35. Ng B., F. Yang, D. P. Huston, Y. Yan, Y. Yang, Z. Xiong, L. E. Peterson, H. Wang, X. F. Yang. 2004. Increased noncanonical splicing of autoantigen transcripts provides the structural basis for expression of untolerized epitopes. J. Allergy Clin. Immunol. 114:1463.
36. Corbet S., H. V. Nielsen, L. Vinner, S. Lauemoller, D. Therrien, S. Tang, G. Kronborg, L. Mathiesen, P. Chaplin, S. Brunak, S. Buus, A. Fomsgaard. 2003. Optimization and immune recognition of multiple novel conserved hla-a2, human immunodeficiency virus type 1-specific ctl epitopes. J. Gen. Virol. 84:2409.
37. Logean A., D. Rognan. 2002. Recovery of known t-cell epitopes by computational scanning of a viral genome. J. Comput. Aided Mol. Des. 16:229.
38. Shirai M., T. Arichi, M. Nishioka, T. Nomura, K. Ikeda, K. Kawanishi, V. H. Engelhard, S. M. Feinstone, J. A. Berzofsky. 1995. Ctl responses of hla-a2.1-transgenic mice specific for hepatitis c viral peptides predict epitopes for ctl of humans carrying hla-a2.1. J. Immunol. 154:2733.
39. Gianfrani C., C. Oseroff, J. Sidney, R. W. Chesnut, A. Sette. 2000. Human memory ctl response specific for influenza a virus is broad and multispecific. Hum. Immunol. 61:438.
40. Schechter I., A. Berger. 1967. On the size of the active site in proteases. I. Papain. Biochem. Biophys. Res. Commun. 27:157.
41. Lohmuller T., D. Wenzler, S. Hagemann, W. Kiess, C. Peters, T. Dandekar, T. Reinheckel. 2003. Toward computer-based cleavage site prediction of cysteine endopeptidases. Biol. Chem. 384:899.
42. Holzhutter H. G., C. Frommel, P. M. Kloetzel. 1999. A theoretical approach towards the identification of cleavage-determining amino acid motifs of the 20 s proteasome. J. Mol. Biol. 286:1251.
43. Tenzer S., B. Peters, S. Bulik, O, Schoor, C. Lemmel, M. M. Schatz, P. M. Kloetzel, H. G. Rammensee, H. Schild, H. G. Holzhutter. 2005. Modeling the mhc class i pathway by combining predictions of proteasomal cleavage, tap transport and mhc class i binding. Cell Mol. Life. Sci. 62:1025.
44. Kesmir C., A. K. Nussbaum, H. Schild, V. Detours, S. Brunak. 2002. Prediction of proteasome cleavage motifs by neural networks. Protein Eng. 15:287.
45. Bertsekas D. P., and John N. Tsitsiklis. 2002. Introduction to probability, Athena Scientific Publishing Corp. Nashua.
46. Rosner B. 2000. Hypothesis testing: One-sample inference. B. Rosner ed. Duxbury Publishing Corp. Australia, Canada, Mexico, Singapore, Spain, United Kingdom, United States, pp 211
47. Ellington A. A. C. J. 1997. Characteristics of amino acids. In Current Protocols in Molecular Biology. F. M. Ausubel et al. ed. John Wiley & Sons, Inc., Indianapolis, pp A.1C.1
48. Knipe D., and P. M. Howley. 2001. Fundamental virology. Lippincott Williams & Wilkins Publishing Corp. Philadelphia, Baltimore, New York, London, Buenos Aires, Hong Kong, Sydney, Tokyo.
49. Rosner B. 2000 Estimation of sample size and power for comparing two means. In Fundamentals of Biostatistics. B. Rosner ed. Duxbury Publishing Corp. Australia, Canada, Mexico, Singapore, Spain, United Kingdom, United States, pp
50. Akashi H. 2001. Gene expression and molecular evolution. Curr. Opin. Genet. Dev. 11:660.
51. Agris P. F. 2004. Decoding the genome: A modified view. Nucleic Acids Res. 32:223.
52. Falk K., O. Rotzschke, S. Stevanovic, G. Jung, H. G. Rammensee. 1991. Allele-specific motifs revealed by sequencing of self-peptides eluted from mhc molecules. Nature 351:290.
53. Hunt D. F., R. A. Henderson, J. Shabanowitz, K. Sakaguchi, H. Michel, N. Sevilir, A. L. Cox, E. Appella, V. H. Engelhard. 1992. Characterization of peptides bound to the class i mhc molecule hla-a2.1 by mass spectrometry. Science 255:1261.
54. Parker K. C., M. A. Bednarek, J. E. Coligan. 1994. Scheme for ranking potential hla-a2 binding peptides based on independent binding of individual peptide side-chains. J. Immunol. 152:163.
55. Ruppert J., J. Sidney, E. Celis, R. T. Kubo, H. M. Grey, A. Sette. 1993. Prominent role of secondary anchor residues in peptide binding to hla-a2.1 molecules. Cell 74:929.
56. Madden D. R., D. N. Garboczi, D. C. Wiley. 1993. The antigenic identity of peptide-mhc complexes: A comparison of the conformations of five viral peptides presented by hla-a2. Cell 75:693.
57. Nussbaum A. K., C. Kuttler, K. P. Hadeler, H. G. Rammensee, H. Schild. 2001. Paproc: A prediction algorithm for proteasomal cleavages available on the www. Immunogenetics 53:87.
58. Rammensee H., J. Bachmann, N. P. Emmerich, O. A. Bachor, S. Stevanovic. 1999. Syfpeithi: Database for mhc ligands and peptide motifs. Immunogenetics 50:213.
59. Daniel S., S. Caillat-Zucman, J. Hammer, J. F. Bach, P. M. Van Endert. 1997. Absence of functional relevance of human transporter associated with antigen processing polymorphism for peptide selection. J. Immunol. 159:2350.
60. Doytchinova I. A., V. A. Walshe, N. A. Jones, S. E. Gloster, P. Borrow, D. R. Flower. 2004. Coupling in silico and in vitro analysis of peptide-rnhc binding: A bioinformatic approach enabling prediction of superbinding peptides and anchorless epitopes. J. Immunol. 172:7495.
61. Doytchinova I. A., D. R. Flower. 2002. Quantitative approaches to computational vaccinology. Immunol. Cell Biol. 80:270.
62. Van Den Eynde B. J., S. Morel. 2001. Differential processing of class-irestricted epitopes by the standard proteasome and the immunoproteasome. Curr. Opin. Immunol. 13:147.
63. Rajkumar S. V., P. G. Richardson, T. Hideshima, K. C. Anderson. 2005. Proteasome inhibition as a novel therapeutic target in human cancer. J. Clin. Oncol. 23:630.
64. Goldrath A. W., M. J. Bevan. 1999. Selecting and maintaining a diverse t-cell repertoire. Nature 402:255.
65. Surh C. D., J. Sprent. 2000. Homeostatic t cell proliferation: How far can t cells be activated to self-ligands? J. Exp. Med. 192:F9.
66. Ehrlich R. 1997. Modulation of antigen processing and presentation by persistent virus infections and in tumors. Hum. Immunol. 54:104.
67. Whitby F. G., E. I. Masters, L. Kramer, J. R. Knowlton, Y. Yao, C. C. Wang, C. P. Hill. 2000. Structural basis for the activation of 20 s proteasomes by 11 s regulators. Nature 408:115.
68. Seliger B., U. Wollscheid, F. Momburg, T. Blankenstein, C. Huber. 2001. Characterization of the major histocompatibility complex class i deficiencies in b16 melanoma cells. Cancer Res. 61:1095.
69. Tomkinson B. 1999. Tripeptidyl peptidases: Enzymes that count. Trends Biochem. Sci. 24:355.
70. Fruci D., G. Lauvau, L. Saveanu, M. Amicosante, R. H. Butler, A. Polack, F. Ginhoux, F. Lemonnier, H. Firat, P. M. Van Endert. 2003. Quantifying recruitment of cytosolic peptides for hla class i presentation: Impact of tap transport. J. Immunol. 170:2977.
71. Knuehl C., P. Spee, T. Ruppert, U. Kuckelkorn, P. Henklein, J. Neefjes, P. M. Kloetzel. 2001. The murine cytomegalovirus pp 89 immunodominant h-21d epitope is generated and translocated into the endoplasmic reticulum as an 11-mer precursor peptide. J. Immunol. 167:1515.
72. Fruci D., G. Niedermann, R. H. Butler, P. M. Van Endert. 2001. Efficient mhc class i-independent amino-terminal trimming of epitope precursor peptides in the endoplasmic reticulum. Immunity 15:467.
73. Saric T., S. C. Chang, A. Hattori, I. A. York, S. Markant, K. L. Rock, M. Tsujimoto, A. L. Goldberg. 2002. An ifn-gamma-induced aminopeptidase in the er, erap1, trims precursors to mhc class i-presented peptides. Nat. Immunol. 3:1169.
74. Bonehill A., C. Heirman, K. Thielemans. 2005. Genetic approaches for the induction of a cd4+t cell response in cancer immunotherapy. J. Gene Med. 7:686.

While the invention has been described in detail and with reference to specific examples thereof, it will be apparent to one skilled in the art that various changes and modifications can be made therein without departing from the spirit and scope thereof.

What is claimed is:

1. A method for characterizing a tumor antigen to serve as the antigen for the generation of tumor specific vaccines capable of eliciting a T cell immune response comprising:
(a) identifying HLA restricted T cell reactive epitopes;
(b) identifying proteolytic cleavage sites of the HLA restricted T cell reactive epitopes;
(c) statistically analyzing the identified immunodominant tumor antigen epitopes to determine whether they are statistically different from those of known documented immunodominant viral epitopes;
wherein a tumor antigen statistically different from those of known documented immunodominant viral epitopes can serve as the antigen for the generation of tumor specific vaccines capable of eliciting T cell immune response.

2. A method of selecting a tumor antigen to serve as the antigen for the generation of tumor specific vaccines capable of eliciting a T cell immune response comprising:
(a) identifying HLA restricted T cell reactive epitopes;
(b) identifying proteolytic cleavage sites of the HLA restricted T cell reactive epitopes;
(c) statistically analyzing the identified immunodominant tumor antigen epitopes to determine whether they are statistically different from those of known documented immunodominant viral epitopes;
(d) selecting the identified immunodominant tumor antigen epitopes which are statistically different from those of known documented immunodominant viral epitopes;
wherein a tumor antigen statistically different from those of known documented immunodominant viral epitopes can serve as the antigen for the generation of tumor specific vaccines capable of eliciting T cell immune response.

3. A method for the generation of tumor specific vaccines capable of eliciting a T cell immune response comprising:
(a) identifying HLA restricted T cell reactive epitopes;
(b) identifying proteolytic cleavage sites of the HLA restricted T cell reactive epitopes;
(c) statistically analyzing the identified immunodominant tumor antigen epitopes to determine whether they are statistically different from those of known documented immunodominant viral epitopes;
(d) selecting the identified immunodominant tumor antigen epitopes which are statistically different from those of known documented immunodominant viral epitopes;
(e) making a pharmaceutical composition comprising one or more of the identified immunodominant tumor antigen epitopes as a tumor specific vaccine capable of eliciting a T cell immune response;
(f) administering the pharmaceutical composition;
thereby eliciting a T cell immune response.

4. The method of claim 3, wherein the pharmaceutical composition further comprises one or more members selected from the group consisting of a cytokine, a chemotherapeutic agent, a chemokine, and an adjuvant.

5. The method of claim 4, wherein said cytokine is selected from the group consisting of a tumor necrosis factor, an interleukin, a lymphokine, granulocyte colony-stimulating factor (G-CSF), a granulocyte macrophage colony-stimulating factor (GM-CSF), a macrophage colony-stimulating factor (M-CSF), monocyte chemoattractant protein 1 (CP1), macrophage inflammatory protein MIP1α, macrophage inflammatory protein MIP1β, IL-1, IL-2, IL-3, IL4, IL-5, IL-6, IL-7, IL-8, IL-9, IL-10, IL-11, IL-12, IL-13, IL-14, IL-15, IL-16, IL-17, IL-18, TNF-α, IFN-α, IFN-γ, and IL-20 (MDA-7).

6. A method of treatment of cancer by administration of tumor specific vaccines capable of eliciting a T cell immune response comprising:
(a) identifying HLA restricted T cell reactive epitopes;
(b) identifying proteolytic cleavage sites of the HLA restricted T cell reactive epitopes;
(c) statistically analyzing the identified immunodominant tumor antigen epitopes to determine whether they are statistically different from those of known documented immunodominant viral epitopes;
(d) selecting the identified immunodominant tumor antigen epitopes which are statistically different from those of known documented immunodominant viral epitopes;
(e) making a pharmaceutical composition comprising one or more of the identified immunodominant tumor antigen epitopes as a tumor specific vaccine capable of eliciting a T cell immune response;
(f) administering the pharmaceutical composition to a patient;
thereby treating the patient.

7. The method of claim 4, wherein the composition comprising the tumor specific vaccine capable of eliciting T cell immune response further comprises one or more members selected from the group consisting of a cytokine, a chemotherapeutic agent, a chemokine, and an adjuvant.

8. The method of claim 5, wherein said cytokine is selected from the group consisting of a tumor necrosis factor, an interleukin, a lymphokine, granulocyte colony-stimulating factor (G-CSF), a granulocyte macrophage colony-stimulating factor (GM-CSF), a macrophage colony-stimulating factor (M-CSF), monocyte chemoattractant protein 1 (CP1), macrophage inflammatory protein MIP1α, macrophage inflammatory protein MIP1β, IL-1, IL-2, IL-3, IL4, IL-5, IL-6, IL-7, IL-8, IL-9, IL-10, IL-11, IL-12, IL-13, IL-14, IL-15, IL-16, IL-17, IL-18, TNF-α, IFN-α, IFN-γ, and IL-20 (MDA-7).

9. An isolated tumor specific vaccine capable of eliciting a T cell immune response identified by the method comprising:
(a) identifying HLA restricted T cell reactive epitopes;
(b) identifying proteolytic cleavage sites of the HLA restricted T cell reactive epitopes;
(c) statistically analyzing the identified immunodominant tumor antigen epitopes to determine whether they are statistically different from those of known documented immunodominant viral epitopes;
(d) selecting the identified immunodominant tumor antigen epitopes which are statistically different from those of known documented immunodominant viral epitopes;
(e) making a pharmaceutical composition comprising one or more of the identified immunodominant tumor antigen epitopes as a tumor specific vaccine capable of eliciting a T cell immune response;
wherein a tumor antigen statistically different from those of known documented immunodominant viral epitopes can serve as the antigen for the generation of tumor specific vaccines capable of eliciting T cell immune response.

10. A tumor specific vaccine capable of eliciting a T cell immune response of claim 7, comprising an epitope as set forth in FIG. 4.

11. A kit comprising the tumor specific vaccine capable of eliciting a T cell immune response of claim 8, further comprising an adjuvant, and a pharmaceutically acceptable carrier.

12. The kit of claim 9 further comprising one or more members selected from the group consisting of a cytokine, a chemotherapeutic agent, a chemokine, and an adjuvant.

13. The kit of claim 10, wherein said cytokine is selected from the group consisting of a tumor necrosis factor, an interleukin, a lymphokine, granulocyte colony-stimulating factor (G-CSF), a granulocyte macrophage colony-stimulating factor (GM-CSF), a macrophage colony-stimulating factor (M-CSF), monocyte chemoattractant protein 1 (CP1), macrophage inflammatory protein MIP1α, macrophage inflammatory protein MIP1β, IL-1, IL-2, IL-3, IL4, IL-5, IL-6, IL-7, IL-8, IL-9, IL-10, IL-11, IL-12, IL-13, IL-14, IL-15, IL-16, IL-17, IL-18, TNF-α, IFN-α, IFN-γ, and IL-20 (MDA-7).
